SEQUENCE LISTING (37 sequences)

SEQ ID NO: 1 - DNA, 355 bp, Homo sapiens
caggtacagctgcagcagtcaggtccaggactcgtgaagccctcgcagaccctctcactcacctgtggcatctccggggacagtgtctctagcacgcgcgctgcttggaattggatcaggcagtccccatcgagaggccttgagtggctgggaaggacatactacaggtccaagtggtattatgattatgcagtctctgtgaaaggtcgaataaccttcaccccagacacatccaagaaccaggtctccctgcacctgaacgctgtgactcccgaggacacggctatgtattactgtgtaaggggcaatatttttgatgtgtggggccaagggacaatggtcaccgtctcaagcg

SEQ ID NO: 2 - protein, 118 aa, Homo sapiens
QVQLQQSGPGLVKPSQTLSLTCGISGDSVSSTRAAWNWIRQSPSRGLEWLGRTYYRSKWYYDYAVSVKGRITFTPDTSKNQVSLHLNAVTPEDTAMYYCVRGNIFDVWGQGTMVTVSS

SEQ ID NO: 3 - protein, 10 aa, Homo sapiens
GDSVSSTRAA

SEQ ID NO: 4 - protein, 18 aa, Homo sapiens
RTYYRSKWYYDYAVSVKG

SEQ ID NO: 5 - protein, 6 aa, Homo sapiens
GNIFDV

SEQ ID NO: 6 - DNA, 333 bp, Homo sapiens
aattttatgctgactcagccccattcggtgtcggagtctccggggaagacggtgaccatctcctgcacccgcaacagtggcaacattgccaccgcctatgtgcagtggtaccaacagcgcccgggcagttcccccaccaccgtgatctttcaggattttcaaagaccctctggggtccctgatcgcttctcaggctccatcgacaggtcctccaattctgcctccctcaccatctctggcctaaagactgaggacgaggctgattactattgtcagtcttatgataacaactatcgcgctgttttcggaggaggcaccctgctgaccgtcctc

SEQ ID NO: 7 - protein, 111 aa, Homo sapiens
NFMLTQPHSVSESPGKTVTISCTRNSGNIATAYVQWYQQRPGSSPTTVIFQDFQRPSGVPDRFSGSIDRSSNSASLTISGLKTEDEADYYCQSYDNNYRAVFGGGTLLTVL

SEQ ID NO: 8 - protein, 14 aa, Homo sapiens
CTRNSGNIATAYVQ

SEQ ID NO: 9 - protein, 7 aa, Homo sapiens
QDFQRPS

SEQ ID NO: 10 - protein, 10 aa, Homo sapiens
QSYDNNYRAV

SEQ ID NO: 11 - DNA, 354 bp, Homo sapiens
caggtacagctgcagcagtcaggtccaggactggtgaagccctcgcagaccctctcactcacctgtgccatctccggggacagtgtctctagcaagaattcttcttggaactggatcaggcagtccccatcgagaggccttgagtggctggggaggacatactacaggtccaagtggtattatgattatgcagtctctgtgaaaggtcgaataaccttcaccccagacacatccaagaaccaggtctccctgcacctgaacgctgtgactcccgaggacacggctatgtattactgtgtaaggggcaatatttttgatgtgtggggccaagggacaatggtcaccgtctcaagc

SEQ ID NO: 12 - protein, 118 aa, Homo sapiens
QVQLQQSGPGLVKPSQTLSLTCAISGDSVSSKNSSWNWIRQSPSRGLEWLGRTYYRSKWYYDYAVSVKGRITFTPDTSKNQVSLHLNAVTPEDTAMYYCVRGNIFDVWGQGTMVTVSS

SEQ ID NO: 13 - protein, 12 aa, Homo sapiens
GDSVSSKNSSWN

SEQ ID NO: 14 - protein, 18 aa, Homo sapiens
RTYYRSKWYYDYAVSVKG

SEQ ID NO: 15 - protein, 6 aa, Homo sapiens
GNIFDV

SEQ ID NO: 16 - DNA, 400 bp, Homo sapiens
  variation (383)...(383): n can be A, C, T or G
cggaccgcttccatccactcctgtgtcttctctacaggcgtgcacagccttaattttatgctgactcagccccactctgtgtcggagtctccggggaagacggtgaccatctcctgcacccgcaacagtggcaacattgccaccgcctatgtgcagtggtaccaacagcgcccgggcagttcccccaccactgtgatctttcaggattttcaaagaccctctggggtccctgatcgcttctcaggctccatcgacaggtcctccaattctgcctccctcaccatctctggcctaaagactgaggacgaggctgattactattgtcagtcttatgataacaactatcgcgctgttttcggaggaggcaccctgctgaccgtccnccgtgagtagcggtccg

SEQ ID NO: 17 - protein, 127 aa, Homo sapiens
RTASIHSCVFSTGVHSLNFMLTQPHSVSESPGKTVTISCTRNSGNIATAYVQWYQQRPGSSPTTVIFQDFQRPSGVPDRFSGSIDRSSNSASLTISGLKTEDEADYYCQSYDNNYRAVFGGGTLLTV

SEQ ID NO: 18 - protein, 13 aa, Homo sapiens
TRNSGNIATAYVQ

SEQ ID NO: 19 - protein, 7 aa, Homo sapiens
QDFQRPS

SEQ ID NO: 20 - protein, 10 aa, Homo sapiens
QSYDNNYRAV

SEQ ID NO: 21 - DNA, 366 bp, Homo sapiens
caggtgcagctggtgcagtctgggggaggcgtggtccagcctgggaggtccctgagactctcctgtgcagcctctggattcaccttcagtagctatgctatgcactgggtccgccaggctccaggcaaggggttggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagatcccgaggggtggccggaaaaggggatgcttttgatatctggggccaagggacaaaggtcaccgtctcaagc

SEQ ID NO: 22 - protein, 122 aa, Homo sapiens
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARSRGVAGKGDAFDIWGQGTKVTVSS

SEQ ID NO: 23 - protein, 10 aa, Homo sapiens
GFTFSSYAMH

SEQ ID NO: 24 - protein, 17 aa, Homo sapiens
VISYDGSNKYYADSVKG

SEQ ID NO: 25 - protein, 12 aa, Homo sapiens
SRGVAGKGDAFD

SEQ ID NO: 26 - DNA, 394 bp, Homo sapiens
cggaccgcttccatccactcctgtgtcttctctacaggcgtgcacagccttgatgttgtgatgactcagtctccactctccctgcccgtcacccctggagagccggcctccatctcctgcaggtctagtcagagcctcctgcatagtaatggatacaactatttggattggtatctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaggtctacaaactccgtacacttttggccaggggaccaagctggagatcaaacgtgagt

SEQ ID NO: 27 - protein, 131 aa, Homo sapiens
RTASIHSCVFSTGVHSLDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGLQTPYTFGQGTKLEIKRE

SEQ ID NO: 28 - protein, 16 aa, Homo sapiens
RSSQSLLHSNGYNYLD

SEQ ID NO: 29 - protein, 7 aa, Homo sapiens
LGSNRAS

SEQ ID NO: 30 - protein, 9 aa, Homo sapiens
MQGLQTPYT

SEQ ID NO: 31 - protein, 9 aa, Artificial Sequence
  peptide epitope
SLYNTVATL

SEQ ID NO: 32 - protein, 9 aa, Artificial Sequence
  peptide epitope
RLRAEAQVK

SEQ ID NO: 33 - protein, 8 aa, Artificial Sequence
  peptide epitope
RYPLTFGW

SEQ ID NO: 34 - protein, 7 aa, Artificial Sequence
  peptide epitope
LPFDKTM

SEQ ID NO: 35 - protein, 9 aa, Artificial Sequence
  peptide epitope
RPMTYKAAL

SEQ ID NO: 36 - protein, 9 aa, Artificial Sequence
  peptide epitope
SRYWAIRTR

SEQ ID NO: 37 - protein, 9 aa, Artificial Sequence
  peptide epitope
RRIYDLIEL

(19) United States
(12) Patent Application Publication
(10) Pub. No.: US 2013/0315933 A1
(43) Pub. Date: Nov. 28, 2013

(54) Antibodies Directed Against HLA-B27 Homodimers and Methods and Uses Thereof in Diagnosis and Therapy

(71) Applicants: Christoph Renner, Zurich (CH); Andreas Wadle, Zurich (CH); Sravan Payeli, Zurich (CH); Markus Thiel, Zurich (CH); Paul Bowness, Oxford (GB); Simon Kollnberger, Oxford (GB)

(72) Inventors: Christoph Renner, Zurich (CH); Andreas Wadle, Zurich (CH); Sravan Payeli, Zurich (CH); Markus Thiel, Zurich (CH); Paul Bowness, Oxford (GB); Simon Kollnberger, Oxford (GB)

(21) Appl. No.: 13/877,958

(22) PCT Filed: Oct. 6, 2011
(86) PCT No.: PCT/US11/01722

§ 371 Date: Jun. 24, 2013

Related U.S. Application Data
(60) Provisional application No. 61/404,614, filed on Oct. 6, 2010.

(51) Int. Cl. C07K 16/28

(52) U.S. Cl. CPC C07K 16/28
USPC 424/173.1; 435/7.24; 435/69.6; 436/501; 530/387.3; 530/388.73; 530/389.6; 530/391.3; 530/391.7; 536/23.53

(57) ABSTRACT

Specific binding members, particularly antibodies and fragments thereof, which bind to HLA-B27 heavy-chain homodimers, termed HC-B27, HLA-B27₂ or B27₂, particularly recognizing B27₂ homodimers and which do not recognize or bind HLA-B27 heterotrimers (B27) including HLA-B27 heterotrimers with β2 microglobulin and peptide. These antibodies are useful in the diagnosis and treatment of HLA-B27 mediated conditions, particularly those associated with B27₂, the spondylarthritides, a group of related diseases including ankylosing spondylitis (AS) and reactive arthritis (ReA or Reiter's syndrome). The antibodies, variable regions or CDR domain sequences thereof, and fragments thereof of the present invention may also be used in therapy in combination with chemotherapeutics, immune modulators, anti-inflammatory drugs, NSAIDs and/or with other antibodies or fragments thereof. Antibodies of this type are exemplified by the novel antibodies HD4, HD5 and HD6 whose sequences are provided herein.

### FIELD OF THE INVENTION

The present invention relates to specific binding members, particularly antibodies and fragments thereof, which bind to HLA-B27 heavy-chain homodimers, termed HC-B27 or B27₂, particularly recognizing B27₂ homodimers and which do not recognize or bind HLA-B27 heterotrimers (B27) including HLA-B27 heterotrimers with β2 microglobulin and peptide. These antibodies are useful in the diagnosis and treatment of HLA-B27 mediated conditions, particularly those associated with B27₂, the spondylarthritides, a group of related diseases including ankylosing spondylitis (AS) and reactive arthritis (ReA or Reiter's syndrome). In addition, the antibodies and fragments thereof can be used for the diagnosis, monitoring and treatment of Spondyloarthritides conditions, such as ankylosing spondylitis. The antibodies, variable regions or CDR domain sequences thereof, and fragments thereof of the present invention may also be used in therapy in combination with chemotherapeutics, immune modulators, anti-inflammatory drugs, NSAIDs and/or with other antibodies or fragments thereof.

### BACKGROUND OF THE INVENTION

Possession of HLA-B27 is strongly associated with development of spondylarthritides, a group of related diseases including ankylosing spondylitis (AS), reactive arthritis (ReA or Reiter's syndrome) (follows infection with species of Chlamydia, Campylobacter, Salmonella, Shigella and Yersinia), sacroileitis associated with psoriasis, sacroileitis associated with inflammatory bowel disease, undifferentiated oligoarthropathy, anterior uveitis, aortic regurgitation together with cardiac conduction abnormality and enthesis-related juvenile idiopathic arthritis (Powness P (2002) Rheumatology 41:857-868), the most recognized being AS and ReA. Clinical features of the spondyloarthropathies include enthesitis (inflammation at sites where tendons, ligaments, or joint capsules attach to bone), inflammatory back pain, dactylitis, and extra-articular manifestations such as uveitis and skin rash. The association of HLA-B27 with ankylosing spondylitis was first described in 1973 (Brewerton D A et al (1973) Lancet i:904-907), and is among the strongest described for a HLA locus, with 94% of AS patients HLA-B27 positive versus 9.4% of controls (Brown M A et al (1996) Ann Rheum Dis 55:268-270). Approximate HLA-B27 frequency is reported as follows with these spondyloarthritides: ankylosing spondylitis 96%; undifferentiated spondylarthropathy 70%; reactive arthritis 30-70%; colitis-associated spondylarthritis 33-75%; psoriatic spondylarthritis 40-50%; juvenile enthesitis-related arthritis 70%; iritis 50%; and cardiac conduction defects with aortic incompetence up to 88% (McMichael A and Bowness P (2002) Arthritis Res 4(suppl):S153-S158) Despite intensive research, the pathogenic role of HLA-B27 remains unclear (for review, see Allen R L et al (1999) Immunogenetics 50:220-227).

The natural immunologic function of HLA-B27 is to bind antigenic peptides together with β2-microglobulin (β2m) for presentation to the T cell receptor (TCR) of CD8+ cytotoxic T lymphocytes. HLA-B27 binds and presents peptides from influenza, HIV, Epstein-Barr virus and other viruses, leading to specific cytotoxic T lymphocyte responses which play an important role in the body's immune responses to these viruses (Townsend A et al (1986) Cell 44:959-968; Gotch F et al (1987) Nature 326:881-882; Bowness P et al (1994) Eur J Immunol 24:2357-2363). However, certain features of disease in HLA-B27 transgenic rat (Hammer R E et al (1990) Cell 63:1099-1112) and mouse (Khare S D et al (1995) J Exp Med 182:1153-1158) models of spondylarthritis have suggested a possible pathogenic role for HLA-B27 heavy chains independent of β2m. Thus, murine disease requires expression of HLA-B27 in the absence of murine β2m (m β2m), and can occur in animals with extremely few CD8+ T cells (Khare S D et al (1995) J Exp Med 182:1153-1158). Furthermore, disease onset is delayed and severity reduced by administration of the monoclonal antibody (mAb) HC-10 (Khare S D et al (1996) J Clin Invest 98:2746; Khare S D et al (1998) J Immunol 160:101-106). HC-10 antibody recognizes free human HLA class I heavy chains (Stam et al (1986) J Immunol 137:2299-2306). These results have led to the suggestion that HLA-B27 heavy chains may be directly involved in disease pathogenesis (Khare S D et al (1995) J Exp Med 182:1153-1158). Disease in the rat requires a high copy number of the HLA-B27 transgene (Taurog J D et al (1993) J Immunol 150:4168-4178), and disease cannot be transferred by CD8+ T cells alone (Breban M et al (1996) J Immunol 156:794-803). Two transgenic rat lines, 33-3 and 21-4H, carrying high gene copy numbers of HLA-B27 and its human β2-microglobulin partner, consistently develop multiorgan inflammation, resembling human HLA-B27-associated disease, with features including colitis, enteritis, peripheral and axial arthritis, male genital inflammation, and psoriform skin and nail lesions (Hammer R E et al (1990) Cell 63:1099).

The formation of β2m-free disulfide-bonded HLA-B27 heavy-chain homodimers, termed HC-B27 or B27₂, have been recently described (Allen R L et al. (1999) J Immunol 162:5045-5048). Dimerization in vitro is dependent on the presence of the free cysteine at position 67 of the HLA-B27 heavy-chain al helix. The β2m-free HLA-B27 heavy chains could also be detected on the surface of HLA-B27-transfected cells (Allen R L et al. (1999) J Immunol 162:5045-5048). Studies in animals show that HLA-B27 transgenic animals express HC10 antibody-reactive HLA-B27 H chains as homodimers and multimers in a variety of lymphoid cells, both intracellularly and at the cell surface (Kollneberger S et al (2004) J Immunol 173:1699-1710). The murine paired Ig-like receptors (PIRs) are ligands for B27₂ in mice, and these receptors share considerable sequence homology (40-60%) with human leukocyte Ig-like receptor (LILR)/leukocyte Ig receptor (LIR) family of receptors (Dennis G et al (1999) J Immunol 163:6371), and a model has been suggested whereby B27₂ expressed by APC in the mice interact with PIRs on monocytes or B cells to induce or perpetuate immunopathology.

In addition to direct cognate interactions with the TCR, mature class I complexes have been shown to bind several other immunomodulatory molecules, including members of the killer cell immunoglobulin-like receptor (KIR) family, and the immunoglobulin-like transcripts (ILT; also known as leukocyte Ig-like receptors, or LIR (Andre P et al (2001) Nat Immunol 2:661)). KIRs are expressed on certain natural killer (NK), T, and NKT cells (for review, see Lanier L L (1998) Cell 92:705-707). KIRs are polymorphic and demonstrate allele-specific recognition, with the cognate KIR for HLA-B27 being the 3-domain KIR3DL1. ILT/LIRs have a somewhat different expression pattern, with ILT2 expressed on B cells, as well as NK, T cells, and monocyte/macrophages (Colonna M et al (1997) J Exp Med 186:1809-1818). ILT4 is more selectively expressed on dendritic cells, monocytes, and macrophages. ILT2 and ILT4 receptor family members have a broader specificity, with ILT2 recognizing all of the class I alleles previously studied (Colonna M et al (1997) J Exp Med 186:1809-1818). ILT4 binds to most HLA-A and B alleles studied, as well as to the nonclassic HLA-G (Colonna M et al (1998) J Immunol 160:3096-3100; Allan D S et al (1999) J Exp Med 189:1149-1156).

Kollnberger et al have shown that both HLA-B27 heavy-chain homodimers and receptors for HLA-B27 homodimers are expressed on populations of peripheral blood and synovial monocytes and B and T lymphocytes from patients with spondylarthritis (Kollnberger S et al. (2002) Arthr & Rheumatol 46(11):2972-2982). Control subjects also express receptors for HLA-B27 heavy-chain homodimers. KIR3DL1, KIR3DL2, and ILT4 and at least one additional receptor, but not ILT2, are capable of binding to HLA-B27 heavy-chain homodimers. These interactions could contribute to joint inflammation and disease pathogenesis in the spondylarthritides.

Thus, there exists a need in the art for a means and methods to specifically assess and evaluate HLA-B27 homodimers and for a specific antibody that recognizes and binds to HLA-B27 homodimers. The availability of a B27₂ specific antibody would provide a means to quantitate, monitor, assess and modulate HLA-B27 homodimers in disease and immunologically and cellularly mediated diseases and conditions which involve these homodimers. Accordingly, it would be desirable to develop B27₂ specific antibodies, particularly antibodies which do not recognize or bind HLA-B27 heterotrimers (B27), including HLA-B27 heterotrimers complexed with β2 microglobulin and peptide, and which demonstrate efficacy and applicability in diagnosis and therapy of HLA-mediated disease or conditions, and it is toward the achievement of that objective that the present invention is directed.

The citation of references herein shall not be construed as an admission that such is prior art to the present invention.

### SUMMARY OF THE INVENTION

In a general aspect, the present invention provides novel antibodies and active fragments thereof directed against the HLA-B27 homodimers B27₂. The specific antibodies of the invention have been utilized to demonstrate the association of B27₂ homodimers with spondylartitides disease, particularly Ankylosing Spondylitis (AS) and to prove the existence of HLA-B27 homodimers on monocytes of Ankylosing Spondylitis patients. In addition, the antibodies of the invention significantly inhibit the interaction of HLA-B27 homodimers with disease-associated immunoreceptors.

The invention provides antibodies directed against HLA-B27 for diagnostic and therapeutic purposes. In particular, antibodies specific for HLA-B27 are provided, wherein said antibodies recognize and are capable of binding specifically to HLA-B27 homodimers B27₂ and which do not recognize other HLA-B27 forms including HLA-B27 heterotrimers (B27) and HLA-B27 heterotrimers with β2 microglobulin and peptide. Thus, in an aspect of the invention, antibodies are provided which are specific for a pathological form of HLA, associated with disease states, and which do not recognize or cross react with physiologically relevant forms of HLA which present peptide and assist in recognition and immunological clearance of agents or pathogens, such as viruses. Active fragments of the antibodies of the invention, particularly Fab antibodies, are provided herein. The antibodies of the present invention have diagnostic and therapeutic use in conditions associated with HLA-B27 mediated conditions, particularly those associated with B27₂, the spondylarthritides, a group of related diseases including ankylosing spondylitis (AS), reactive arthritis (ReA or Reiter's syndrome), sacroileitis associated with psoriasis, sacroileitis associated with inflammatory bowel disease, undifferentiated oligoarthropathy, anterior uveitis, aortic regurgitation together with cardiac conduction abnormality and enthesis-related juvenile idiopathic arthritis. In a particular aspect the antibodies of the invention are applicable in B27₂ mediated disease including ankylosing spondylitis (AS) and reactive arthritis (ReA or Reiter's syndrome).

In a general aspect, the present invention provides HLA-B27 antibodies directed against HLA-B27 heavy-chain homodimers, B27₂, and which do not recognize or bind HLA-B27 heterotrimers (B27) including HLA-B27 heterotrimers with β2 microglobulin and peptide. In a broad aspect, the present invention provides an isolated specific binding member, particularly an antibody or fragment thereof, including a Fab fragment and a single chain or domain antibody, which specifically recognizes HLA-B27 homodimers. In an important aspect of the invention, the antibodies and fragments of the invention specifically recognize HLA-B27 homodimers and do not bind or recognize HLA-B27 heterotrimers, which include HLA-B27 complexed with peptide. In a further aspect, the present invention provides an antibody or fragment thereof, which recognizes HLA-B27 homodimers B27₂ and comprises the heavy and light chain variable region amino acid sequence of antibody selected from HD4, HD5 and HD6 including as set out in FIG. 12 (SEQ ID NO: 2 and 7), FIG. 13 (SEQ ID NO: 12 and 17) and/or FIG. 14 (SEQ ID NO: 22 and 27). In one such aspect, the invention provides an anti-B27₂ antibody comprising the variable region CDR sequences set out in FIG. 12 (SEQ ID NOS: 3-5 and 8-10), FIG. 13 (SEQ ID NOS: 13-15 and 18-20) or FIG. 14 (SEQ ID NOS: 23-25 and 28-30) or in Table 1.

In a particular aspect, the antibody or fragment of the invention is reactive with, capable of specifically binding B27₂ and does not bind other forms of HLA-B27. In a further aspect the antibody or fragment does not react with, does not bind to HLA-B27 heterotrimers (B27) including HLA-B27 heterotrimers with β2 microglobulin and peptide. In an aspect, the antibody or fragment of the invention binds or recognizes B27₂ cell free or cell surface-expressed B27₂. In an aspect of the invention, the antibody or fragment thereof recognizes or binds B27₂ expressed or present on peripheral blood mononuclear cells (PBMCs) or monocytes. In an additional aspect, the antibody or fragment specifically inhibits immunoreceptor recognition of B27₂. In another aspect the antibody or fragment induces, mediates apoptosis in FAP expressing cells. In a still additional aspect the antibody or fragment inhibits or otherwise reduces/blocks HLA-B27 binding to immune cell innate immune receptors, including Killer Immunoglobulin-like Receptors (KIR) and Leukocyte Immunoglobulin-like receptors (LIR). In a further aspect the antibody or fragment inhibits or otherwise reduces/blocks B27₂ binding to KIR3DL1, KIRsDL2 and LILRB2 receptors.

The present inventors have discovered novel B27₂ antibodies which are reactive to HLA-B27 homodimers and do not react with HLA-B27 heterotrimers or HLA-B27 complexed with β2m and/or with peptide. The antibodies exemplified herein include Fab antibodies and recombinant antibodies based thereon. Exemplary antibodies provided include HD4, HD5 and HD6. The antibodies have the heavy and light chain variable region sequences and comprise CDR domain region sequences as set out herein and in FIGS. 12 (SEQ ID NOS: 3-5 and 8-10), 13 (SEQ ID NOS: 13-15 and 18-20) and 14 (SEQ ID NOS: 23-25 and 28-30). In a preferred aspect the antibody of the invention is a monoclonal antibody and the fragment is an Fab fragment. The isolated antibody or fragment of the invention may in the form of an antibody F(ab′)2, scFv fragment, diabody, triabody or tetrabody.

The unique specificity and affinity of the antibodies and fragments of the invention provides diagnostic and therapeutic uses to identify, characterize and target HLA-B27 mediated diseases and conditions, particularly conditions associated with HLA-B27 homodimers, Particularly Spondylarthritides, particularly without the problems associated with normal HLA-B27 heterotrimer or immunologically necessary HLA-B27 form recognition and binding. Thus, use of the antibodies and fragments of the invention avoids cross-reaction with other physiologically important HLA-B27 forms and molecules and any untoward or negative immunological effects associated therewith. The antibodies and fragments provide specific reagents for a pathologically relevant form of HLA-B27. Diseases or conditions facilitated by or associated with the presence or relatively increased levels or amounts of HLA-B27 homodimers B27₂ are particularly susceptible to and targeted by the antibodies of the present invention. Such disease or conditions include the spondylarthritides, particularly ankylosing spondylitis (AS), reactive arthritis (ReA or Reiter's syndrome), sacroileitis, anterior uveitis, aortic regurgitation and juvenile idiopathic arthritis.

In a preferred aspect, the antibody is one which has the characteristics of the antibodies which the inventors have identified and characterized, in particular specifically recognizing B27₂ forms of HLA-B27. In a particularly preferred aspect the antibody is HD4, HD5 or HD6, or active fragments thereof. In a further preferred aspect the antibody of the present invention comprises the VH and VL amino acid sequences depicted in FIGS. 12 (SEQ ID NOS: 2 and 7), 13 (SEQ ID NOS: 12 and 17) and/or 14 (SEQ ID NOS: 22 and 27). In a particular aspect, the antibody of the invention comprises the CDR sequences (CDR1, CDR2, CDR3) depicted in FIG. 12 (SEQ ID NOS: 3-5 and 8-10), 13 (SEQ ID NOS: 13-15 and 18-20) or 14 (SEQ ID NOS: 23-25 and 28-30) or in Table 1. In a particular aspect of the invention the antibody is HD6 and comprises the heavy and light chain variable region sequences set out in FIG. 12 (SEQ ID NOS: 2, 7). In a particular aspect of the invention the antibody is HD6 and comprises the CDR region sequences set out in FIG. 12 (SEQ ID NOS: 3-5, 8-10). In a particular aspect of the invention the antibody is HD4 and comprises the heavy and light chain variable region sequences set out in FIG. 13 (SEQ ID NOS: 12, 17). In a particular aspect of the invention the antibody is HD4 and comprises the CDR region sequences set out in FIG. 13 (SEQ ID NOS: 13-15, 18-20). In a particular aspect of the invention the antibody is HD5 and comprises the heavy and light chain variable region sequences set out in FIG. 14 (SEQ ID NOS: 22, 27). In a particular aspect of the invention the antibody is HD5 and comprises the CDR region sequences set out in FIG. 14 (SEQ ID NOS: 23-25, 28-30).

The binding of an antibody to its target antigen is mediated through the complementarity-determining regions (CDRs) of its heavy and light chains. Accordingly, specific binding members based on the CDR regions of the heavy or light chain, and preferably both, of the antibodies of the invention, particularly of HD4, HD5 and/or HD6, will be useful specific binding members for therapy and/or diagnostics. The sequences and CDRs of the antibodies are depicted in FIGS. 12, 13 and 14 and in Table 1. Antibody HD6 comprises heavy chain CDR sequences GDSVSSTRAA (CDR1) (SEQ ID NO: 3), RTYYRSKWYYDYAVSVKG (CDR2) (SEQ ID NO: 4) and GNIFDV (CDR3) (SEQ ID NO: 5), and light chain CDR sequences CTRNSGNIATAYVQ (CDR1) (SEQ ID NO: 8), QDFQRPS (CDR2) (SEQ ID NO: 9) and QSYDNNYRAV (CDR3) (SEQ ID NO: 10), as set out in FIG. 12. Antibody HD4 comprises heavy chain CDR sequences GDSVSSKNSSWN (CDR1) (SEQ ID NO: 13), RTYYRSKWYYDYAVSVKG (CDR2) (SEQ ID NO: 14) and GNIFDV (CDR3) (SEQ ID NO: 15), and light chain CDR sequences TRNSGNIATAYVQ (CDR1) (SEQ ID NO: 18), QDFQRPS (CDR2) (SEQ ID NO: 19) and QSYDNNYRAV (CDR3) (SEQ ID NO: 20), as set out in FIG. 13. Antibody HD5 comprises heavy chain CDR sequences GFTFSSYAMH (CDR1) (SEQ ID NO: 23), VISYDGSNKYYADSVKG (CDR2) (SEQ ID NO: 24) and SRGVAGKGDAFD (CDR3) (SEQ ID NO: 25), and light chain CDR sequences RSSQSLLHSNGYNYLD (CDR1) (SEQ ID NO: 28), LGSNRAS (CDR2) (SEQ ID NO: 29) and MQGLQTPYT (CDR3) (SEQ ID NO: 30), as set out in FIG. 14.

Accordingly, specific binding proteins such as antibodies which are based on the CDRs of the antibody(ies) identified herein will be useful for targeting HLA-B27, particularly HLA-B27 homodimers B27₂ and HLA-B27 homodimer expressing cells or cells with homodimer on their cell surfaces in diseases or in Spondyloarthritides.

In an aspect of the invention, the isolated antibody or fragment of the invention is an antibody or antibody fragment comprising a heavy chain and a light chain variable region comprising an amino acid sequence selected from the amino acid sequence set out in FIG. 12, 13 or 14, particularly a heavy and light chain comprising the variable region heavy and light chain CDR1, CDR2 and CDR3 sequences set out in FIG. 12, 13 or 14 (SEQ ID NOS: 3-5 and 8-10, SEQ ID NOS: 13-15 and 18-20, SEQ ID NOS: 23-25 and 28-30, respectively), or highly homologous variants thereof comprising 1 to 3 amino acid substitutions in one or more CDR region of FIG. 12, 13 or 14 (SEQ ID NOS: 3-5. 8-10, 13-15, 18-20, 23-25, 28-30), wherein said variants retain B27₂ specific binding. In a further aspect, the present invention provides an isolated antibody or fragment thereof capable of binding an antigen, wherein said antibody or fragment thereof comprises a polypeptide binding domain comprising an amino acid sequence substantially as set out herein and in FIG. 12, 13 or 14 (SEQ ID NO: 2 and 7, SEQ ID NO: 12 and 17, SEQ ID NO: 22 and 27).

In further aspects, the invention provides an isolated nucleic acid which comprises a sequence encoding a specific binding member as defined above, and methods of preparing specific binding members of the invention which comprise expressing said nucleic acids under conditions to bring about expression of said binding member, and recovering the binding member. In one such aspect, a nucleic acid encoding antibody variable region sequence having the amino acid sequences as set out in FIG. 12, 13 or 14 is provided or an antibody having CDR domain sequences as set out in FIG. 12, 13 or 14 is provided (nucleic acid encoding SEQ ID NOS: 2, 7, 3-5 or 8-10, nucleic acid encoding SEQ ID NOS: 12, 17, 13-15 or 18-20, or nucleic acid encoding SEQ ID NOS: 22, 27, 23-25 or 28-30). In one aspect, a nucleic acid of FIG. 12, 13 or 14 is provided (nucleic acid encoding SEQ ID NOS: 2 and 7, 12 and 17, 22 and 27). In one such aspect a nucleic acid encoding a heavy chain variable region sequence is provided which nucleic acid comprises SEQ ID NO: 1, SEQ ID NO: 11 or SEQ ID NO: 21 or relevant CDR region encoding nucleic acids thereof. A nucleic acid encoding a light chain variable region sequence is provided which nucleic acid comprises SEQ ID NO: 6, SEQ ID NO: 16 or SEQ ID NO: 26 or relevant CDR region encoding nucleic acids thereof. The present invention also relates to a recombinant DNA molecule or cloned gene, or a degenerate variant thereof, which encodes an antibody of the present invention; preferably a nucleic acid molecule, in particular a recombinant DNA molecule or cloned gene, encoding the antibody VH and VL, particularly the CDR region sequences, which has a sequence or is capable of encoding a sequence shown in FIG. 12, 13 or 14.

The antibodies, fragments thereof and recombinant antibodies comprising the CDR domains according to the invention may be used in a method of treatment or diagnosis of the human or animal body, such as a method of treatment of a Spondyloarthritidopathy in a human patient which comprises administering to said patient an effective amount of the antibodies, fragments thereof and recombinant antibodies of the invention. In a particular aspect of the invention, antibodies, fragments thereof and recombinant antibodies comprising the CDR domains according to the invention may be used in a method of treatment or amelioration of Ankylosing Spondylitis or reactive arthritis in a mammal which comprises administering to said mammal an effective amount of the antibodies, fragments thereof and recombinant antibodies of the invention.

The diagnostic utility of the present invention extends to the use of the antibodies of the present invention in assays to characterize samples or patients for Spondyloarthritides diseases or conditions, including in vitro and in vivo diagnostic assays. In an immunoassay, a control quantity of the antibodies, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached.

Specific binding members, antibodies, or fragments thereof of the invention may carry a detectable or functional label. The specific binding members may carry a radioactive label, such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re. When radioactive labels are used, known currently available counting procedures may be utilized to identify and quantitate the specific binding members. In the instance where the label is an enzyme, detection may be accomplished by any of the presently utilized colorimetric, spectrophotometric, fluorospectrophotometric, amperometric or gasometric techniques known in the art. The isolated antibody or fragment of the invention may further comprise a detectable or functional label. In an aspect thereof, the detectable or functional label may be a covalently attached drug. In a further aspect, the detectable or functional label may be a radiolabel or an enzyme.

The radiolabelled specific binding members, particularly antibodies and fragments thereof, are useful in in vitro diagnostics techniques and in in vivo radioimaging techniques. In a further aspect of the invention, radiolabelled specific binding members, particularly antibodies and fragments thereof, particularly radioimmunoconjugates, are useful in radioimmunotherapy, particularly as radiolabelled antibodies for cellular therapy.

Immunoconjugates or antibody fusion proteins of the present invention, wherein the specific binding members, particularly antibodies and fragments thereof, of the present invention are conjugated or attached to other molecules or agents further include, but are not limited to binding members conjugated to a chemical ablation agent, toxin, immunomodulator, anti-inflammatory, cytokine, cytotoxic agent, chemotherapeutic agent or drug.

The present invention includes an assay system which may be prepared in the form of a test kit for the quantitative analysis of the extent of the presence of, for instance, HLA-B27 homodimers. The system or test kit may comprise a labeled component prepared by one of the radioactive and/or enzymatic techniques discussed herein, coupling a label to the antibody, and one or more additional immunochemical reagents, at least one of which is a free or immobilized components to be determined or their binding partner(s).

The invention provides a method for diagnosing or monitoring an HLA-B27 mediated disease or condition in a mammal wherein said disease or condition is diagnosed or monitored by determining the presence and/or amount of HLA-B27 homodimer comprising:

A. contacting a biological sample from a mammal in which the presence of and HLA-B27 mediated disease or condition is suspected with the antibody or fragment of the present invention a set out in any of FIG. 12, 13 or 14 (SEQ ID NOS: 2 and 7, SEQ ID NOS: 12 and 17, SEQ ID NOS: 22 and 27), or having the CDR region sequences thereof (SEQ ID NOS: 3-5 and 8-10, SEQ ID NOS: 13-15 and 18-20, SEQ ID NOS: 23-25 and 28-30), under conditions that allow binding of HLA-B27 homodimer to said antibody to occur; and

B. detecting whether binding has occurred between HLA-B27 homodimer from said sample and the antibody or determining the amount of binding that has occurred said HLA-B27 homodimer from said sample and the antibody;

wherein the detection of binding indicates the presence of HLA-B27 homodimer in said sample and of an HLA-B27 mediated disease or condition in said mammal.

In an aspect of the above method, the antibody comprises a heavy chain and light chain variable region comprising an amino acid sequence selected from the amino acid sequence set out in FIG. 12, 13 or 14 (SEQ ID NO: 2 and 7, SEQ ID NO: 12 and 17, SEQ ID NO: 22 and 27), comprising the CDR region CDR1, CDR2 and CDR3 sequences of the heavy and light chain variable region (SEQ ID NOS: 3-5 and 8-10, SEQ ID NOS: 13-15 and 18-20, SEQ ID NOS: 23-25 and 28-30), or highly homologous variants thereof comprising 1 to 3 amino acid substitutions in one or more CDR region of FIG. 12, 13 or 14, wherein said variants retain B27₂ specific binding. In a particular aspect, the method may be utilized for diagnosing or monitoring one or more disease or condition selected from ankylosing spondylitis (AS), reactive arthritis (ReA or Reiter's syndrome), sacroileitis associated with psoriasis, sacroileitis associated with inflammatory bowel disease, undifferentiated oligoarthropathy, anterior uveitis, aortic regurgitation together with cardiac conduction abnormality and enthesis-related juvenile idiopathic arthritis.

A kit is contemplated by the present invention for the diagnosis or prognosis of an HLA-B27 mediated disease in which HLA-B27 homodimer B27₂ is present, said kit comprising an antibody or fragment characterized by having specific binding to HLA-B27 homodimers, optionally with reagents and/or instructions for use.

In a further embodiment, the present invention relates to certain therapeutic methods which would be based upon the activity of the binding member, antibody, or active fragments thereof, or upon agents or other drugs determined to possess the same activity. A first therapeutic method is associated with the prevention or treatment of spondylarthritides, including ankylosing spondylitis (AS), reactive arthritis (ReA or Reiter's syndrome), sacroileitis, undifferentiated oligoarthropathy, anterior uveitis, aortic regurgitation together with cardiac conduction abnormality and enthesis-related juvenile idiopathic arthritis.

The binding members and antibodies of the present invention, and in a particular embodiment the antibody whose sequences are presented in FIGS. 12, 13 and 14 herein, or active fragments thereof, and single chain, recombinant or synthetic antibodies derived therefrom, particularly comprising the CDR region sequences depicted in FIG. 12, 13 or 14 or in Table 1, can be prepared in pharmaceutical compositions, including a suitable vehicle, carrier or diluent, for administration in instances wherein therapy is appropriate, such as to treat cancer. Such pharmaceutical compositions may also include methods of modulating the half-life of the binding members, antibodies or fragments by methods known in the art such as pegylation. Such pharmaceutical compositions may further comprise additional antibodies or therapeutic agents.

A composition of the present invention may be administered alone or in combination with other treatments, therapeutics or agents, either simultaneously or sequentially dependent upon the condition to be treated. In addition, the present invention contemplates and includes compositions comprising the binding member, particularly antibody or fragment thereof, herein described and other agents or therapeutics such as anti-inflammatory or immunomodulatory agents or therapeutics, anti-mitotic agents, apoptotic agents or antibodies, or immune modulators. Other treatments or therapeutics may include the administration of suitable doses of pain relief drugs such as non-steroidal anti-inflammatory drugs (e.g. aspirin, paracetamol, ibuprofen or ketoprofen) or opiates such as morphine, or anti-emetics.

The present invention also includes antibodies and fragments thereof, which are covalently attached to or otherwise associated with other molecules or agents. These other molecules or agents include, but are not limited to, molecules (including antibodies or antibody fragments) with distinct recognition characteristics, toxins, ligands, and chemotherapeutic agents. In an additional aspect the antibodies or fragments of the invention may be used to target or direct therapeutic molecules or other agents, for example to target molecules or agents to HLA-B27 homodimer expressing cells, for example to monocytes or NK cells expressing HLA-B27 homodimers or binders thereof.

Other objects and advantages will become apparent to those skilled in the art from a review of the ensuing detailed description, which proceeds with reference to the following illustrative drawings, and the attendant claims.

### BRIEF DESCRIPTION OF THE DRAWINGS

FIG. 1A-1C. Characterisation of Phage-Derived B27₂-Specific Antibodies

A. ELISA showing binding of phage-derived clones to recombinant B27₂ and B27 heterotrimer (HT). Three different phage-derived clones (HD4, HD5 and HD6) demonstrated B27₂ specific binding. Irr=irrelevant ‘phage (n=3). B. SDS PAGE analysis of HD6 Fab and IgG molecules under reducing (left) and non-reducing (right) conditions. C. HD6 binding pattern to different recombinant HLA-A2, -B7, -B27 and B27₂ complexes in comparison to HC10, ME1, W6/32 and BB7.2 antibodies in ELISA (n=6).

FIGS. 2A and 2B. Recognition of Cell-Surface Expressed B272 by HD6

A. Flow cytometric (FACS) analysis of HD6 binding to untransfected LBL721.220 cells or LBL721.220 cells transfected with HLA-B7, B27 C67S (greatly reduced or absent B27₂), B27 HuTPN (reduced B27₂ and increased B27), or B27₂ (dimer expressing) as indicated. Each panel shows IgG1 isotype control, ME1 (IgG1), w6/32 (IgG2a), HC10 (IgG2a) and HD6 (IgG1) staining performed at equal concentrations on equal numbers of cells in a single experiment (n=4). B. Western Blot for the detection of HA-tagged HLA-B27 dimer after immuno-precipitation (IP) of HLA-B27 transduced U937 monocytic cell lysates. IP samples were analyzed on SDS-PAGE under non-reducing and reducing conditions (with DTT) before Western Blotting for HA tag.

FIG. 3A-D. B27₂ Expression by PBMC from HLA-B27+ Healthy Individuals and AS Patients

A. B27₂ expression on PBMC derived monocytes C. and B-lymphocytes were compared among B27− healthy controls (HC B27−, left panel), B27+ healthy controls (HC B27+, middle panel) and B27+ AS patients (AS B27+, right panel). Isotype controls (IgG1 & IgG2a), HC10 and ME1 antibodies were used as control antibodies and representative histograms are presented {HC-B27− (n=7), HC-B27+ (n=4) and AS-B27+ (n=7)}. B. Histogram showing the average mean fluorescence intensities (MFI) of PBMC derived monocytes D. and B-lymphocytes for all samples. The respective mean values±SEM for B are: HC B27− 0.3±0.1; HC B27+ 1.5±0.6; AS B27+ 12.2±3.3. The respective mean values±SEM for D are: HC B27− 0.1±0.03; HC B27+ 1.7±0.3; AS B27+ 1.9±0.5. p-values were calculated using a one-tailed unpaired t-test with welch's correction are depicted.

FIGS. 4A and 4B. HD6 Inhibited B27₂ Binding to KIR3DL1, KIR3DL2 and LILRB2 Receptors

A. Baf3 cells over-expressing KIR3DL1 (left panel), KIR3DL2 (middle panel) or LILRB2 (right panel) were either stained with PE-labelled B27₂ (homodimer, upper panel) or heterotrimer (HT, lower panel) tetramer in the presence of IgG, HD6 or HC10. B. HD6 pre-incubation inhibited 33.60±5.7% (n=5, p=0.0006), whereas HC10 inhibited 69.4±8.5 (n=5, p<0.0001) of total binding to KIR3DL2 receptor expressing cells.

FIG. 5A-5C. HD6 Inhibits the Effects of Co-Culture of KIR3DL2+ Human NK Cells with B27₂ Expressing Cells (Protection from Apoptosis and Inhibition of IFNγ Production)

A. KIR3DL2+ (upper panel) and KIR3DL2− (lower panel) hYT NK cells were co-cultured for 72 hours with irradiated LBL721.220 B27 (expressing B27₂) or control cells in the presence of HD6 or IgG1 control antibody. Apoptotic cells were identified by double staining with annexin V and live dead Pacific blue. B. Total numbers of KIR3DL2+ or − hYT NK cells (after gating for pacific blue and annexin V) after co-culture with B27₂ expressing cells. For blocking experiments, LBL.721.220 cells expressing B27₂ were pre-incubated with HD6 or IgG1 isotype control MAb (10 μg/ml). Input cell number was 50,000. Mean and sd of triplicate estimations shown; representative of 3 experiments. C. IFNγ levels (determined by ELISA) after co-culturing of KIR3DL2+ hYT NK cells with LBL721.220 B27 for 12 hours in the presence IgG, HD6 or HC10. * p<0.05, ** p<0.01.

FIGS. 6A and 6B. Use of Recombinant Proteins for Selection and Chimeric Antibody Generation

A. Cartoon illustrating recombinant HLA-B27 heterotrimer (heavy chain, β2m and peptide, left) and homodimer (two heavy chains, absence of β2m & peptides, right) used for antibody selection. Note the B27 heavy chain intra-cytoplasmic domain has been deleted and substituted by 6 histidines and a biotinylation recognition sequence. The 6×His tag enabled purification and biotinylation permitted immobilisation and tetramer generation. Bound peptide is indicated in the HLA-B27 complex but may be absent in B27₂. B. Capture ELISA confirming the chimeric phenotype of HD6. Anti-mouse Fc capture antibody was used as coating antibody, followed by anti-human Fab-HRP as detection antibody. Anti-human Fc antibody, mouse and human antibodies served as controls (n=3).

FIGS. 7A and 7B. HD6 has a Different Binding Specificity Compared to HC10

A. Recombinant B27₂ protein was treated with 10 mM DTT at indicated intervals and Western Blot was performed under non-reducing conditions. Homodimer bands were detected by HD6 antibody as described. B. Competition between HD6 and HC10 binding for B27₂ in ELISA. B27 homodimer was pre-incubated either with HD6 or HC10 in excess amounts and the complex was allowed for binding on HD6 or HC10 coated wells. Experiments were performed in triplicates and representative of three independent experiments are shown.

FIGS. 8A and 8B. HD6 and HC10 have Comparable Affinity and Avidity for B27₂

A. Dissociation constant (Kd) was determined using Fab fragments of HD6 (upper panel) and HC10 (lower panel) over immobilized B27₂ in surface Plasmon resonance. Concentrations used were, from upper to lower traces, 8 μM, 4 μM, 2 μM, 1 μM, 500 nM and 0 moles for HD6, and 6.8 μM, 3.4 μM, 1.7 μM, 0.85 μM, 0.45 μM and 0 moles for HC10. Representative of three independent experiments are indicated. B. IgG affinity for homodimer was determined using serially 5 fold diluted HD6 or HC10 at 100 μg/ml-1 pg/ml (666 μM-0.0006 nM) on 1 μg/L of immobilized B27₂. Representative of three independent experiments were shown. Estimated affinity constants of HD6 and HC10 Fabs are indicated in the table. ME1 (IgG1) served as irrelevant control.

FIGS. 9A and 9B. B27₂ Quantification and HD6 Binding Specificity

A. Semi-quantitative measurement of HD6 staining to LBL721.220 B27 cells was performed using Quantibrite beads as described. LBL721.220 B7 cells and W6/32 antibody served as controls. B. Representative staining of LBL721.220 and LBL721.221 cells expressing different HLA molecules with ME1, W6/32, HC10 and HD6 antibodies (n=3).

FIGS. 10A and 10B. HD6 Staining of Monocytes from AS PBMCs and Synovial Fluid

A. Forward and side scatter gated monocytes and lymphocytes were further gated for CD14 and HD6 positivity by co-staining. Gating of peripheral blood CD14 monocytes from an AS patient is shown. B. Comparison of HD6 staining of paired synovial fluid mononuclear cells (SFM) and peripheral blood CD 14+ monocytes from two HLA-B27+ SpA patients.

FIG. 11. Culture in the Presence of B27₂-Expressing 0.220B27 Cells Inhibits Apoptosis of KIR3DL2+ Natural Killer Cells Ex Vivo; this Effect is Partially Blocked by HD6.

5 day culture ex vivo of KIR3DL2+ (upper panels) and KIR3DL2− (lower panels). hYT NK cells were co-cultured for 72 hours with irradiated LBL721.220 B27 (expressing B27₂) or control cells in the presence of HD6 or IgG 1 control antibody. Apoptotic cells were identified by double staining with annexin V and live dead Pacific blue.

FIGS. 12A and 12B depicts the HD6 antibody sequence. (A) Heavy chain cDNA (SEQ ID NO: 1) and amino acid sequence (SEQ ID NO: 2). CDR regions (SEQ ID NOS: 3-5) are depicted in color in the amino acid sequence. CDR1 (SEQ ID NO: 3) is shown in red, CDR2 (SEQ ID NO: 4) is shown in green, and CDR3 (SEQ ID NO: 5) is shown in blue. (B) Light chain cDNA (SEQ ID NO: 6) and amino acid sequence (SEQ ID NO: 7). CDR regions (SEQ ID NOS: 8-10) are depicted in color in the amino acid sequence. CDR1 (SEQ ID NO: 8) is shown in red, CDR2 (SEQ ID NO: 9) is shown in green, and CDR3 (SEQ ID NO: 10) is shown in blue.

FIGS. 13A and 13B depicts the HD4 antibody sequence. (A) Heavy chain cDNA (SEQ ID NO: 11) and amino acid sequence (SEQ ID NO: 12). CDR regions (SEQ ID NOS: 13-15) are depicted in color in the amino acid sequence. CDR1 (SEQ ID NO: 13) is shown in red, CDR2 (SEQ ID NO: 14) is shown in green, and CDR3 (SEQ ID NO: 15) is shown in blue. (B) Light chain cDNA (SEQ ID NO: 16) and amino acid sequence (SEQ ID NO: 17). CDR regions (SEQ ID NOS: 18-20) are depicted in color in the amino acid sequence. CDR1 (SEQ ID NO: 18) is shown in red, CDR2 (SEQ ID NO: 19) is shown in green, and CDR3 (SEQ ID NO: 20) is shown in blue.

FIGS. 14A and 14B depicts the HD5 antibody sequence. (A) Heavy chain cDNA (SEQ ID NO: 21) and amino acid sequence (SEQ ID NO: 22). CDR regions (SEQ ID NOS: 23-25) are depicted in color in the amino acid sequence. CDR1 (SEQ ID NO: 23) is shown in red, CDR2 (SEQ ID NO: 24) is shown in green, and CDR3 (SEQ ID NO: 25) is shown in blue. (B) Light chain cDNA (SEQ ID NO: 26) and amino acid sequence (SEQ ID NO: 27). CDR regions (SEQ ID NOS: 28-30) are depicted in color in the amino acid sequence. CDR1 (SEQ ID NO: 28) is shown in red, CDR2 (SEQ ID NO: 29) is shown in green, and CDR3 (SEQ ID NO: 30) is shown in blue.

FIG. 15A-15D. HD6 an Anti-HLA-B27₂ Specific IgG Antibody

A. Immobilization of HLA-B27₂ homodimer & HLA-B27 heterotrimer to a streptavidin coated gold chip shows binding of the HD6 antibody of HLA-B27₂ homodimer but not to HLA-B27 heterotrimer or empty flow cell by SPR. B. HD6 binds to HLA-B27₂ homodimers with high avidity (KD=2.8 nM). Increasing concentrations of HD6 were flowed over the immobilised homodimer. C. ELISA specificity of HD6 and control antibodies tested against different recombinant HLA class I complexes (A1, B7, B13, C7, B27 & B27₂) show the specific binding of HD6 to HLA-B27₂.homodimers (n=4). D. Western blot analysis reveals that HD6 binds specifically to HLA-B27₂ homodimers but not to HLA-B27 heterotrimers. HD6 & HD10 recognize monomeric forms of HLA-B27 as β2m-free heavy chains following DTT treatment.

FIG. 16. Recognition of Cell-Surface HLA-B27₂ Homodimers by HD6

Representative DAB stained sections of LBL721.220 and LBL721.220 B27 cells. HD6 (upper panel) HD10 (central panel) and W6/32 (lower panel) antibodies show the positive staining of HLA-B27₂ homodimers. Magnification 40×, scale bar 20 μm C. Western Blot for the detection of HA-tagged HLA-B27₂ homodimers and HLA-B27 monomers after immuno-precipitation (IP) of HLA-B27 transduced U937 monocytic cell lysates. IP samples were analyzed on SDS-PAGE under non-reducing and reducing conditions (with DTT) before Western Blotting for HA tag.

FIG. 17. Characterisation of HLA-B27₂ Specific Antibodies by Direct ELISA

Direct ELISA against HLA-G and HLA-B27₂ homodimers (n=4) using HD6, HD10 and W6/32 antibodies.

FIG. 18. HD6 Staining does not Cross-React with Tissues from Human Healthy Patients

Representative DAB stained sections form tissue arrays obtained from human healthy patients (Biochain). Isotype control antibody (left panel), HD6 antibody (central panel) and control HD10 (left panel) were tested. Magnification 20×, scale bar 40 μm. LBL721.220 and LBL721.220 B27 cells were used as positive controls (bottom panels). Magnification 40×, scale bar 50 μm.

FIGS. 19A and 19B. Characteristics of Fisher 33-3 HLA-B27 Transgenic Rats

A) Spontaneous onset of disease in Fischer ‘33-3’ HLA-B27 transgenic rats. Onset of diarrhea, peripheral arthritis, genital inflammation & psoriasis is expressed in % of affected rats and weeks of age. B) Body weight gain with time for control rats (F344) and transgenic rats (HLA-B27).

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Therefore, if appearing herein, the following terms shall have the definitions set out below.

### A. TERMINOLOGY

The term “specific binding member” describes a member of a pair of molecules which have binding specificity for one another. The members of a specific binding pair may be naturally derived or wholly or partially synthetically produced. One member of the pair of molecules has an area on its surface, or a cavity, which specifically binds to and is therefore complementary to a particular spatial and polar organisation of the other member of the pair of molecules. Thus the members of the pair have the property of binding specifically to each other. Examples of types of specific binding pairs are antigen-antibody, biotin-avidin, hormone-hormone receptor, receptor-ligand, enzyme-substrate. This application is concerned with antigen-antibody type reactions.

The term “antibody” describes an immunoglobulin whether natural or partly or wholly synthetically produced. The term also covers any polypeptide or protein having a binding domain which is, or is homologous to, an antibody binding domain. CDR grafted antibodies are also contemplated by this term. An “antibody” is any immunoglobulin, including antibodies and fragments thereof, that binds a specific epitope. The term encompasses polyclonal, monoclonal, and chimeric antibodies, the last mentioned described in further detail in U.S. Pat. Nos. 4,816,397 and 4,816,567. The term “antibody(ies)” includes a wild type immunoglobulin (Ig) molecule, generally comprising four full length polypeptide chains, two heavy (H) chains and two light (L) chains, or an equivalent Ig homologue thereof (e.g., a camelid nanobody, which comprises only a heavy chain); including full length functional mutants, variants, or derivatives thereof, which retain the essential epitope binding features of an Ig molecule, and including dual specific, bispecific, multispecific, and dual variable domain antibodies; Immunoglobulin molecules can be of any class (e.g., IgG, IgE, IgM, IgD, IgA, and IgY), or subclass (e.g., IgG1, IgG2, IgG3, IgG4, IgA1, and IgA2). Also included within the meaning of the term “antibody” are any “antibody fragment”.

An “antibody fragment” means a molecule comprising at least one polypeptide chain that is not full length, including (i) a Fab fragment, which is a monovalent fragment consisting of the variable light (VL), variable heavy (VH), constant light (CL) and constant heavy 1 (CH1) domains; (ii) a F(ab′)2 fragment, which is a bivalent fragment comprising two Fab fragments linked by a disulfide bridge at the hinge region; (iii) a heavy chain portion of an Fab (Fd) fragment, which consists of the VH and CH1 domains; (iv) a variable fragment (Fv) fragment, which consists of the VL and VH domains of a single arm of an antibody, (v) a domain antibody (dAb) fragment, which comprises a single variable domain (Ward, E. S. et al., Nature 341, 544-546 (1989)); (vi) a camelid antibody; (vii) an isolated complementarity determining region (CDR); (viii) a Single Chain Fv Fragment wherein a VH domain and a VL domain are linked by a peptide linker which allows the two domains to associate to form an antigen binding site (Bird et al, Science, 242, 423-426, 1988; Huston et al, PNAS USA, 85, 5879-5883, 1988); (ix) a diabody, which is a bivalent, bispecific antibody in which VH and VL domains are expressed on a single polypeptide chain, but using a linker that is too short to allow for pairing between the two domains on the same chain, thereby forcing the domains to pair with the complementarity domains of another chain and creating two antigen binding sites (WO94/13804; P. Holliger et al Proc. Natl. Acad. Sci. USA 90 6444-6448, (1993)); and (x) a linear antibody, which comprises a pair of tandem Fv segments (VH-CH1-VH-CH1) which, together with complementarity light chain polypeptides, form a pair of antigen binding regions; (xi) multivalent antibody fragments (scFv dimers, trimers and/or tetramers (Power and Hudson, J Immunol. Methods 242: 193-204 9 (2000)); and (xii) other non-full length portions of heavy and/or light chains, or mutants, variants, or derivatives thereof, alone or in any combination.

As antibodies can be modified in a number of ways, the term “antibody” should be construed as covering any specific binding member or substance having a binding domain with the required specificity. Thus, this term covers antibody fragments, derivatives, functional equivalents and homologues of antibodies, including any polypeptide comprising an immunoglobulin binding domain, whether natural or wholly or partially synthetic. Chimeric molecules comprising an immunoglobulin binding domain, or equivalent, fused to another polypeptide are therefore included. Cloning and expression of chimeric antibodies are described in EP-A-0120694 and EP-A-0125023 and U.S. Pat. Nos. 4,816,397 and 4,816,567.

An “antibody combining site” is that structural portion of an antibody molecule comprised of light chain or heavy and light chain variable and hypervariable regions that specifically binds antigen.

The phrase “antibody molecule” in its various grammatical forms as used herein contemplates both an intact immunoglobulin molecule and an immunologically active portion of an immunoglobulin molecule.

Exemplary antibody molecules are intact immunoglobulin molecules, substantially intact immunoglobulin molecules and those portions of an immunoglobulin molecule that contains the paratope, including those portions known in the art as Fab, Fab′, F(ab′)₂ and F(v), which portions are preferred for use in the therapeutic methods described herein.

Antibodies may also be bispecific, wherein one binding domain of the antibody is a specific binding member of the invention, and the other binding domain has a different specificity, e.g. to recruit an effector function or the like. Bispecific antibodies of the present invention include wherein one binding domain of the antibody is a specific binding member of the present invention, including a fragment thereof, and the other binding domain is a distinct antibody or fragment thereof, including that of a distinct immune or blood cell specific antibody. The other binding domain may be an antibody that recognizes or targets a particular cell type, as in a PBMC, T cell or monocyte-specific antibody. In the bispecific antibodies of the present invention the one binding domain of the antibody of the invention may be combined with other binding domains or molecules which recognize particular cell receptors and/or modulate cells in a particular fashion, as for instance an immune modulator (e.g., interleukin(s)), a growth modulator or cytokine (e.g. tumor necrosis factor (TNF), and particularly, the TNF bispecific modality demonstrated in U.S. Ser. No. 60/355,838 filed Feb. 13, 2002 incorporated herein in its entirety) or a toxin (e.g., ricin) or anti-mitotic or apoptotic agent or factor. Thus, the anti-B27₂ antibodies of the invention may be utilized to direct or target agents, labels, other molecules or compounds or antibodies to cells expressing or demonstrating HLA-B27 homodimers.

The phrase “monoclonal antibody” in its various grammatical forms refers to an antibody having only one species of antibody combining site capable of immunoreacting with a particular antigen. A monoclonal antibody thus typically displays a single binding affinity for any antigen with which it immunoreacts. A monoclonal antibody may also contain an antibody molecule having a plurality of antibody combining sites, each immunospecific for a different antigen; e.g., a bispecific (chimeric) monoclonal antibody.

The term “antigen binding domain” describes the part of an antibody which comprises the area which specifically binds to and is complementary to part or all of an antigen. Where an antigen is large, an antibody may bind to a particular part of the antigen only, which part is termed an epitope. An antigen binding domain may be provided by one or more antibody variable domains. Preferably, an antigen binding domain comprises an antibody light chain variable region (VL) and an antibody heavy chain variable region (VH).

Immunoconjugates or antibody fusion proteins of the present invention, wherein the antibodies, antibody molecules, or fragments thereof, of use in the present invention are conjugated or attached to other molecules or agents further include, but are not limited to such antibodies, molecules, or fragments conjugated to a chemical ablation agent, toxin, immunomodulator, cytokine, cytotoxic agent, chemotherapeutic agent, antimicrobial agent or peptide, cell wall and/or cell membrane disrupter, or drug.

The term “specific” may be used to refer to the situation in which one member of a specific binding pair will not show any significant binding to molecules other than its specific binding partner(s). The term is also applicable where e.g. an antigen binding domain is specific for a particular epitope which is carried by a number of antigens, in which case the specific binding member carrying the antigen binding domain will be able to bind to the various antigens carrying the epitope.

The term “comprise” generally used in the sense of include, that is to say permitting the presence of one or more features or components.

The term “consisting essentially of” refers to a product, particularly a peptide sequence, of a defined number of residues which is not covalently attached to a larger product. In the case of the peptide of the invention referred to above, those of skill in the art will appreciate that minor modifications to the N- or C-terminal of the peptide may however be contemplated, such as the chemical modification of the terminal to add a protecting group or the like, e.g. the amidation of the C-terminus.

The term “isolated” refers to the state in which specific binding members of the invention, or nucleic acid encoding such binding members will be, in accordance with the present invention. Members and nucleic acid will be free or substantially free of material with which they are naturally associated such as other polypeptides or nucleic acids with which they are found in their natural environment, or the environment in which they are prepared (e.g. cell culture) when such preparation is by recombinant DNA technology practised in vitro or in vivo. Members and nucleic acid may be formulated with diluents or adjuvants and still for practical purposes be isolated—for example the members will normally be mixed with gelatin or other carriers if used to coat microtitre plates for use in immunoassays, or will be mixed with pharmaceutically acceptable carriers or diluents when used in diagnosis or therapy.

As used herein, “pg” means picogram, “ng” means nanogram, “ug” or “μg” mean microgram, “mg” means milligram, “ul” or “μl” mean microliter, “ml” means milliliter, “l” means liter.

The terms “antibody”, “anti-B27₂ antibody”, “HLA-B27 homodimer antibody”, “HLA-B27₂ antibody”, “antibody HD6”, “antibody HD4”, “antibody HD5” and any variants not specifically listed, may be used herein interchangeably, and as used throughout the present application and claims refer to proteinaceous material including single or multiple proteins, and extends to those proteins having the amino acid sequence data described herein and presented in FIGS. 12, 13 and 14 (SEQ ID NOS: 2 and 7, 12 and 17, 22 and 27, or SEQ ID NOS: 3-5, 8-10, SEQ ID NOS: 13-15, 18-20, SEQ ID NOS: 23-25, 28-30) and the profile of activities set forth herein and in the Claims. Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “antibody”, “anti-B27₂ antibody”, “HLA-B27 homodimer antibody”, “HLA-B27₂ antibody”, “antibody HD6”, “antibody HD4”, “antibody HD5” are intended to include within their scope proteins specifically recited herein as well as all substantially homologous analogs and allelic variations.

The amino acid residues described herein are preferred to be in the “L” isomeric form. However, residues in the “D” isomeric form can be substituted for any L-amino acid residue, as long as the desired functional property of immunoglobulin-binding is retained by the polypeptide. NH₂ refers to the free amino group present at the amino terminus of a polypeptide. COOH refers to the free carboxy group present at the carboxy terminus of a polypeptide. In keeping with standard polypeptide nomenclature, J. Biol. Chem., 243:3552-59 (1969), abbreviations for amino acid residues are shown in the following Table of Correspondence:

TABLE OF CORRESPONDENCE

| SYMBOL | | |
|---|---|---|
| 1-Letter | 3-Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus. A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease S1), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

The term “oligonucleotide,” as used herein in referring to the probe of the present invention, is defined as a molecule comprised of two or more ribonucleotides, preferably more than three. Its exact size will depend upon many factors which, in turn, depend upon the ultimate function and use of the oligonucleotide.

The term “primer” as used herein refers to an oligonucleotide, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be either single-stranded or double-stranded and must be sufficiently long to prime the synthesis of the desired extension product in the presence of the inducing agent. The exact length of the primer will depend upon many factors, including temperature, source of primer and use of the method. For example, for diagnostic applications, depending on the complexity of the target sequence, the oligonucleotide primer typically contains 15-25 or more nucleotides, although it may contain fewer nucleotides.

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementarity with the sequence of the strand to hybridize therewith and thereby form the template for the synthesis of the extension product.

As used herein, the terms “restriction endonucleases” and “restriction enzymes” refer to bacterial enzymes, each of which cut double-stranded DNA at or near a specific nucleotide sequence.

A cell has been “transformed” by exogenous or heterologous DNA when such DNA has been introduced inside the cell. The transforming DNA may or may not be integrated (covalently linked) into chromosomal DNA making up the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

Two DNA sequences are “substantially homologous” when at least about 75% (preferably at least about 80%, and most preferably at least about 90 or 95%) of the nucleotides match over the defined length of the DNA sequences. Sequences that are substantially homologous can be identified by comparing the sequences using standard software available in sequence data banks, or in a Southern hybridization experiment under, for example, stringent conditions as defined for that particular system. Defining appropriate hybridization conditions is within the skill of the art. See, e.g., Maniatis et al., supra; DNA Cloning, Vols. I & II, supra; Nucleic Acid Hybridization, supra.

It should be appreciated that also within the scope of the present invention are DNA sequences encoding specific binding members (antibodies) of the invention which code for e.g. an antibody having the same amino acid sequence as provided in FIG. 12, 13 or 14 (SEQ ID NO: 2 and 7, 12 and 17, 22 and 27), or comprising the CDR domain region sequences set out herein or in FIG. 12, 13 or 14 (SEQ ID NOS: 3-5 and 8-10, 13-15 and 18-20, 23-25 and 28-30), but which are degenerate thereto. By “degenerate to” is meant that a different three-letter codon is used to specify a particular amino acid. It is well known in the art that the following codons can be used interchangeably to code for each specific amino acid:

| | |
|---|---|
| Phenylalanine (Phe or F) | UUU or UUC |
| Leucine (Leu or L) | UUA or UUG or CUU or CUC or CUA or |
| | CUG |
| Isoleucine (Ile or I) | AUU or AUC or AUA |
| Methionine (Met or M) | AUG |
| Valine (Val or V) | GUU or GUC of GUA or GUG |
| Serine (Ser or S) | UCU or UCC or UCA or UCG or AGU or |
| | AGC |
| Proline (Pro or P) | CCU or CCC or CCA or CCG |
| Threonine (Thr or T) | ACU or ACC or ACA or ACG |
| Alanine (Ala or A) | GCU or GCG or GCA or GCG |
| Tyrosine (Tyr or Y) | UAU or UAC |
| Histidine (His or H) | CAU or CAC |
| Glutamine (Gln or Q) | CAA or CAG |
| Asparagine (Asn or N) | AAU or AAC |
| Lysine (Lys or K) | AAA or AAG |
| Aspartic Acid (Asp or D) | GAU or GAC |
| Glutamic Acid (Glu or E) | GAA or GAG |
| Cysteine (Cys or C) | UGU or UGC |
| Arginine (Arg or R) | CGU or CGC or CGA or CGG or AGA or |
| | AGG |
| Glycine (Gly or G) | GGU or GGC or GGA or GGG |
| Tryptophan (Trp or W) | UGG |
| Termination codon | UAA (ochre) or UAG (amber) or UGA (opal) |

It should be understood that the codons specified above are for RNA sequences. The corresponding codons for DNA have a T substituted for U.

Mutations can be made in the sequences encoding the amino acids, antibody fragments, CDR region sequences set out in FIG. 12, 13 or 14, or in the heavy and/or light chain variable region sequences of FIGS. 12, 12 and/or 14, such that a particular codon is changed to a codon which codes for a different amino acid. Such a mutation is generally made by making the fewest nucleotide changes possible. A substitution mutation of this sort can be made to change an amino acid in the resulting protein in a non-conservative manner (for example, by changing the codon from an amino acid belonging to a grouping of amino acids having a particular size or characteristic to an amino acid belonging to another grouping) or in a conservative manner (for example, by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein.

The following is one example of various groupings of amino acids:

Amino Acids with Nonpolar R Groups

### Alanine, Valine, Leucine, Isoleucine, Proline, Phenylalanine, Tryptophan, Methionine

Amino Acids with Uncharged Polar R Groups

### Glycine, Serine, Threonine, Cysteine, Tyrosine, Asparagine, Glutamine

Amino Acids with Charged Polar R Groups (Negatively Charged at pH 6.0)
Aspartic acid, Glutamic acid

### Basic Amino Acids (Positively Charged at pH 6.0)

### Lysine, Arginine, Histidine (at pH 6.0)

Another grouping may be those amino acids with phenyl groups:

### Phenylalanine, Tryptophan, Tyrosine

Another grouping may be according to molecular weight (i.e., size of R groups):

| | | | |
|---|---|---|---|
| Glycine | 75 | Alanine | 89 |
| Serine | 105 | Proline | 115 |
| Valine | 117 | Threonine | 119 |
| Cysteine | 121 | Leucine | 131 |
| Isoleucine | 131 | Asparagine | 132 |
| Aspartic acid | 133 | Glutamine | 146 |
| Lysine | 146 | Glutamic acid | 147 |
| Methionine | 149 | Histidine (at pH 6.0) | 155 |
| Phenylalanine | 165 | Arginine | 174 |
| Tyrosine | 181 | Tryptophan | 204 |

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Exemplary and preferred conservative amino acid substitutions include any of: glutamine (Q) for glutamic acid (E) and vice versa; leucine (L) for valine (V) and vice versa; serine (S) for threonine (T) and vice versa; isoleucine (I) for valine (V) and vice versa; lysine (K) for glutamine (Q) and vice versa; isoleucine (I) for methionine (M) and vice versa; serine (S) for asparagine (N) and vice versa; leucine (L) for methionine (M) and vice versa; lysine (L) for glutamic acid (E) and vice versa; alanine (A) for serine (S) and vice versa; tyrosine (Y) for phenylalanine (F) and vice versa; glutamic acid (E) for aspartic acid (D) and vice versa; leucine (L) for isoleucine (I) and vice versa; lysine (K) for arginine (R) and vice versa.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues (preferably at least about 80%, and most preferably at least about 90 or 95%) are identical, or represent conservative substitutions. The CDR regions of two antibodies are substantially homologous when one or more amino acids are substituted with a similar or conservative amino acid substitution, and wherein the antibody/antibodies have the profile of binding and activities of one or more of the antibodies disclosed herein, including particularly the antibodies HD4, HD5 or HD6.

A “heterologous” region of the DNA construct is an identifiable segment of DNA within a larger DNA molecule that is not found in association with the larger molecule in nature. Thus, when the heterologous region encodes a mammalian gene, the gene will usually be flanked by DNA that does not flank the mammalian genomic DNA in the genome of the source organism. Another example of a heterologous coding sequence is a construct where the coding sequence itself is not found in nature (e.g., a cDNA where the genomic coding sequence contains introns, or synthetic sequences having codons different than the native gene). Allelic variations or naturally-occurring mutational events do not give rise to a heterologous region of DNA as defined herein.

A DNA sequence is “operatively linked” to an expression control sequence when the expression control sequence controls and regulates the transcription and translation of that DNA sequence. The term “operatively linked” includes having an appropriate start signal (e.g., ATG) in front of the DNA sequence to be expressed and maintaining the correct reading frame to permit expression of the DNA sequence under the control of the expression control sequence and production of the desired product encoded by the DNA sequence. If a gene that one desires to insert into a recombinant DNA molecule does not contain an appropriate start signal, such a start signal can be inserted in front of the gene.

The term “standard hybridization conditions” refers to salt and temperature conditions substantially equivalent to 5×SSC and 65° C. for both hybridization and wash. However, one skilled in the art will appreciate that such “standard hybridization conditions” are dependent on particular conditions including the concentration of sodium and magnesium in the buffer, nucleotide sequence length and concentration, percent mismatch, percent formamide, and the like. Also important in the determination of “standard hybridization conditions” is whether the two sequences hybridizing are RNA-RNA, DNA-DNA or RNA-DNA. Such standard hybridization conditions are easily determined by one skilled in the art according to well known formulae, wherein hybridization is typically 10-20° C. below the predicted or determined Tₘ with washes of higher stringency, if desired.

The term ‘agent’ means any molecule, including polypeptides, antibodies, polynucleotides, chemical compounds and small molecules. In particular the term agent includes compounds such as test compounds or drug candidate compounds.

The term ‘agonist’ refers to a ligand that stimulates the receptor the ligand binds to in the broadest sense.

The term ‘assay’ means any process used to measure a specific property of a compound. A ‘screening assay’ means a process used to characterize or select compounds based upon their activity from a collection of compounds.

The term ‘preventing’ or ‘prevention’ refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term ‘prophylaxis’ is related to and encompassed in the term ‘prevention’, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

‘Therapeutically effective amount’ means that amount of a drug, compound, antimicrobial, antibody, or pharmaceutical agent that will elicit the biological or medical response of a subject that is being sought by a medical doctor or other clinician. In particular, with regard to an inflammatory disease or condition, the term “effective amount” is intended to include an effective amount of a compound or agent that will bring about a biologically meaningful decrease in the amount of or extent of inflammation or physical discomfort, pain, rash, swelling associated with the disease or condition, for instance. The phrase “therapeutically effective amount” is used herein to mean an amount sufficient to prevent, and preferably reduce by at least about 30 percent, more preferably by at least 50 percent, most preferably by at least 90 percent, a clinically significant change in the condition, such as AS or ReA or other spondyloarthritic condition, or other feature of pathology such as for example, elevated HLA-B27 homodimers, inflammatory cytokine or cell count as may attend its presence and activity.

The term ‘treating’ or ‘treatment’ of any disease or infection refers, in one embodiment, to ameliorating the disease or infection (i.e., arresting the disease or extent or inflammation, pain or arthritis or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or infection, either physically, (e.g., stabilization of a discernible symptom), physiologically, (e.g., stabilization of a physical parameter), or both. In a further embodiment, ‘treating’ or ‘treatment’ relates to slowing the progression of a disease or reducing an infection or inflammatory response.

The phrase “pharmaceutically acceptable” refers to molecular entities and compositions that are physiologically tolerable and do not typically produce an allergic or similar untoward reaction, such as gastric upset, dizziness and the like, when administered to a mammal, particularly a human.

As used herein, “pg” means picogram, “ng” means nanogram, “ug” or “μg” mean microgram, “mg” means milligram, “ul” or “μl” mean microliter, “ml” means milliliter, “l” means liter.

### B. DETAILED DISCLOSURE

The invention provides antibodies directed against HLA-B27 homodimers (B27₂ or HLA-B27₂) for diagnostic and therapeutic purposes. In particular, antibodies specific for HLA-B27 are provided, wherein said antibodies recognize and are capable of binding specifically to HLA-B27 homodimers B27₂ and which do not recognize other HLA-B27 forms including HLA-B27 heterotrimers (B27) and HLA-B27 heterotrimers with β2 microglobulin and peptide. Antibodies are provided which are specific for a pathological form of HLA, associated with disease states, and which do not recognize or cross react with physiologically relevant forms of HLA which present peptide and assist in recognition and immunological clearance of agents or pathogens, such as viruses. The antibodies of the present invention have diagnostic and therapeutic use in conditions associated with HLA-B27 mediated conditions, particularly those associated with B27₂, the spondylarthritides, a group of related diseases including ankylosing spondylitis (AS), reactive arthritis (ReA or Reiter's syndrome), sacroileitis associated with psoriasis, sacroileitis associated with inflammatory bowel disease, undifferentiated oligoarthropathy, anterior uveitis, aortic regurgitation together with cardiac conduction abnormality and enthesis-related juvenile idiopathic arthritis. In a particular aspect the antibodies of the invention are applicable in B27₂ mediated disease including ankylosing spondylitis (AS) and reactive arthritis (ReA or Reiter's syndrome).

In a general aspect, the present invention provides antibodies specific for HLA-B27 are provided, wherein said antibodies recognize and are capable of binding specifically to HLA-B27 homodimers B27₂ and which do not recognize other HLA-B27 forms including HLA-B27 heterotrimers (B27). In a broad aspect, the present invention provides an isolated specific binding member, particularly an antibody or fragment thereof, including an Fab fragment and a single chain or domain antibody, which recognizes B27₂. In a further aspect, the present invention provides an antibody or fragment thereof, which recognizes HLA-B27 homodimers specifically and comprises the amino acid sequence of HD6, HD4 or HD5 including as set out in FIG. 12 (SEQ ID NO: 2 and 7), FIG. 13 (SEQ ID NO: 12 and 17) and/or FIG. 14 (SEQ ID NO: 22 and 27). In one such aspect, the invention provides an anti-B27₂ antibody comprising the variable region CDR sequences set out in FIG. 12 (SEQ ID NOS: 3-5 and 8-10), FIG. 13 (SEQ ID NOS: 13-15 and 18-20) or FIG. 14 (SEQ ID NOS: 23-25 and 28-30) or in Table 1.

The invention provides an antibody or fragment thereof which recognizes HLA-B27 homodimers specifically and comprises the heavy and light chain variable region amino acid sequence as set out in FIG. 12 and in SEQ ID NOS: 2 and 7. The invention includes an antibody or fragment thereof having a heavy chain and light chain or fragment thereof, and comprising the CDR1, 2 and 3 region heavy chain sequences of SEQ ID NOS: 3-5 and the CDR 1, 3 and 3 region light chain sequences of SEQ ID NOS: 8-10. The invention provides antibody HD-6 having the heavy and light chain variable region sequences of SEQ ID NO: 2 and 7, or comprising the heavy chain CDR sequences SEQ ID NOS: 3-5 and the light chain variable region CDR sequences SEQ ID NOS: 8-10.

The invention provides an antibody or fragment thereof which recognizes HLA-B27 homodimers specifically and comprises the heavy and light chain variable region amino acid sequence as set out in FIG. 13 and in SEQ ID NOS: 12 and 17. The invention includes an antibody or fragment thereof having a heavy chain and light chain or fragment thereof, and comprising the CDR1, 2 and 3 region heavy chain sequences of SEQ ID NOS: 13-15 and the CDR 1, 3 and 3 region light chain sequences of SEQ ID NOS: 18-20. The invention provides antibody HD-4 having the heavy and light chain variable region sequences of SEQ ID NO: 12 and 17, or comprising the heavy chain CDR sequences SEQ ID NOS: 13-15 and the light chain variable region CDR sequences SEQ ID NOS: 18-20.

The invention provides an antibody or fragment thereof which recognizes HLA-B27 homodimers specifically and comprises the heavy and light chain variable region amino acid sequence as set out in FIG. 14 and in SEQ ID NOS: 22 and 27. The invention includes an antibody or fragment thereof having a heavy chain and light chain or fragment thereof, and comprising the CDR1, 2 and 3 region heavy chain sequences of SEQ ID NOS: 23-25 and the CDR 1, 3 and 3 region light chain sequences of SEQ ID NOS: 28-30. The invention provides antibody HD-5 having the heavy and light chain variable region sequences of SEQ ID NO: 22 and 27, or comprising the heavy chain CDR sequences SEQ ID NOS: 23-25 and the light chain variable region CDR sequences SEQ ID NOS: 28-30.

The present invention provides an antibody or fragment thereof specific for HLA-B27, wherein said antibody or fragment recognizes and is capable of binding specifically to HLA-B27 homodimers B27₂ and does not recognize or bind other HLA-B27 forms including HLA-B27 heterotrimers (B27) and HLA-B27 heterotrimers with β2 microglobulin and peptide, wherein the antibody or fragment has:

(a) a heavy chain variable domain comprising the CDR1, CDR2 and CDR3 region sequences as set forth in FIG. 12 (SEQ ID NOS: 3-5), and a light chain variable domain comprising the CDR1, CDR2 and CDR3 region sequences as set forth in FIG. 12 (SEQ ID NOS: 8-10);

(b) a heavy chain variable domain comprising the CDR1, CDR2 and CDR3 region sequences as set forth in FIG. 13 (SEQ ID NOS: 13-15), and a light chain variable domain comprising the CDR1, CDR2 and CDR3 region sequences as set forth in FIG. 13 (SEQ ID NOS: 18-20); or

(c) a heavy chain variable domain comprising the CDR1, CDR2 and CDR3 region sequences as set forth in FIG. 14 (SEQ ID NOS: 23-25), and a light chain variable domain comprising the CDR1, CDR2 and CDR3 region sequences as set forth in FIG. 14 (SEQ ID NOS: 28-30).

The invention includes an antibody, or active fragment thereof, characterized by its ability to bind to HLA-B27 homodimers B27₂ wherein said antibody or fragment does not recognize or bind other HLA-B27 forms including HLA-B27 heterotrimers (B27) and HLA-B27 heterotrimers with β2 microglobulin and peptide, wherein the antibody or fragment has:

(a) a heavy chain variable domain having the amino acid sequence of HD-6 as set forth in FIG. 12 (SEQ ID NO: 2), and a light chain variable domain having the amino acid sequence of HD-6 as set forth in FIG. 12 (SEQ ID NO: 7);

(b) a heavy chain variable domain having the amino acid sequence of HD-4 as set forth in FIG. 13 (SEQ ID NO: 12), and a light chain variable domain having the amino acid sequence of HD-4 as set forth in FIG. 13 (SEQ ID NO: 17); or

(c) a heavy chain variable domain having the amino acid sequence of HD-5 as set forth in FIG. 14 (SEQ ID NO: 22), and a light chain variable domain having the amino acid sequence of HD-5 as set forth in FIG. 14 (SEQ ID NO: 27).

Panels of monoclonal antibodies recognizing HLA-B27 homodimers can be screened for various properties; i.e., isotype, epitope, affinity, etc. Of particular interest are antibodies that mimic the activity of exemplary antibodies HD4, HD5 and HD6, and have affinity for HLA-B27 homodimers and do not recognize or bind to other forms of HLA-B27, including HLA-B27 heterotrimers. Such antibodies can be readily identified and/or screened in specific binding member activity assays.

In general, the CDR regions, comprising amino acid sequences substantially as set out as the CDR regions of FIG. 12, 13 or 14 (SEQ ID NOS: 3-5, 8-10, 13-15, 18-20, 23-25, 28-30) will be carried in a structure which allows for binding of the CDR regions to HLA-B27 homodimer B27₂.

The following Table sets out the CDR domain sequences CDR1, CDR2, and CDR3 for each of the exemplary antibodies HD4, HD5 and HD6. Amino acid similarities and differences in the antibody CDR region sequences are evident from the below TABLE 1.

TABLE 1
Antibody CDR Sequences
Heavy Chain

| Antibody | CDR 1 | CDR 2 | CDR 3 |
|---|---|---|---|
| HD4 | GDSVSSKNSSWN | RTYYRSKWYYDYAVSVKG | GNIFDV |
| | (SEQ ID NO: 13) | (SEQ ID NO: 14) | (SEQ ID NO: 15) |
| HD5 | GFTFSSYAMH | VISYDGSNKYYADSVKG | SRGVAGKGDAFD |
| | (SEQ ID NO: 23) | (SEQ ID NO: 24) | (SEQ ID NO: 25) |
| HD6 | GDSVSSTRAA | RTYYRSKWYYDYAVSVKG | GNIFDV |
| | (SEQ ID NO: 3) | (SEQ ID NO: 4) | (SEQ ID NO: 5) |

Light Chain

| Antibody | CDR 1 | CDR 2 | CDR 3 |
|---|---|---|---|
| HD4 | TRNSGNIATAYVQ | QDFQRPS | QSYDNNYRAV |
| | (SEQ ID NO: 18) | (SEQ ID NO: 19) | (SEQ ID NO: 20) |
| HD5 | RSSQSLLHSNGYNYLD | LGSNRAS | MQGLQTPYT |
| | (SEQ ID NO: 28) | (SEQ ID NO: 29) | (SEQ ID NO: 30) |
| HD6 | CTRNSGNIATAYVQ | QDFQRPS | QSYDNNYRAV |
| | (SEQ ID NO: 8) | (SEQ ID NO: 9) | (SEQ ID NO: 10) |

By “substantially as set out” it is meant that that variable region sequences, and/or particularly the CDR sequences, of the invention will be either identical or highly homologous to the specified regions of FIG. 12, 13 or 14 or SEQ ID NOS: 2 and 7, 12 and 17 and 22 and 27, or SEQ ID NOS: 3-5, and 8-10, 13-15 and 18-20, or 23-25 and 28-30. By “highly homologous” it is contemplated that only a few substitutions, preferably from 1 to 8, preferably from 1 to 5, preferably from 1 to 4, or from 1 to 3, or 1 or 2 substitutions may be made in the variable region sequence and/or in the CDR sequences. The term substantially set out as includes particularly conservative amino acid substitutions which do not materially or significantly affect the specificity and/or activity of the instant antibodies. Conservative amino acid substitutions are exemplified herein and also in FIGS. 12, 13 and 14 for the CDR region sequences.

Substitutions may be made in the variable region sequence outside of the CDRs so as to retain the CDR sequences. Thus, changes in the variable region sequence or alternative non-homologous or veneered variable region sequences may be introduced or utilized, such that the CDR sequences are maintained and the remainder of the variable region sequence may be substituted.

Alternatively, substitutions may be made particularly in the CDRs. CDR sequences for exemplary antibodies of the present invention are set out and described herein including in FIGS. 12, 13 and 14 and in Table 1. Antibody HD6 comprises heavy chain CDR sequences GDSVSSTRAA (CDR1) (SEQ ID NO:3), RTYYRSKWYYDYAVSVKG (CDR2) (SEQ ID NO: 4) and GNIFDV (CDR3) (SEQ ID NO: 5), and light chain CDR sequences CTRNSGNIATAYVQ (CDR1) (SEQ ID NO: 8), QDFQRPS (CDR2) (SEQ ID NO: 9) and QSYDNNYRAV (CDR3) (SEQ ID NO: 10), as set out in FIG. 12. Antibody HD4 comprises heavy chain CDR sequences GDSVSSKNSSWN (CDR1) (SEQ ID NO: 13), RTYYRSKWYYDYAVSVKG (CDR2) (SEQ ID NO: 14) and GNIFDV (CDR3) (SEQ ID NO: 15), and light chain CDR sequences TRNSGNIATAYVQ (CDR1) (SEQ ID NO: 18), QDFQRPS (CDR2) (SEQ ID NO: 19) and QSYDNNYRAV (CDR3) (SEQ ID NO: 20), as set out in FIG. 13. Antibody HD5 comprises heavy chain CDR sequences GFTFSSYAMH (CDR1) (SEQ ID NO: 23), VISYDGSNKYYADSVKG (CDR2) (SEQ ID NO: 24) and SRGVAGKGDAFD (CDR3) (SEQ ID NO: 25), and light chain CDR sequences RSSQSLLHSNGYNYLD (CDR1) (SEQ ID NO: 28), LGSNRAS (CDR2) (SEQ ID NO: 29) and MQGLQTPYT (CDR3) (SEQ ID NO: 30), as set out in FIG. 14.

Antibodies of the invention having substitutions as above described and contemplated are selected to maintain the activities and specificity commensurate with the exemplary antibodies, including antibodies HD4, HD5 and HD6 and having the characteristics as set out herein and in the claims.

The structure for carrying the CDRs of the invention will generally be of an antibody heavy or light chain sequence or substantial portion thereof in which the CDR regions are located at locations corresponding to the CDR region of naturally occurring VH and VL antibody variable domains encoded by rearranged immunoglobulin genes. The structures and locations of immunoglobulin variable domains may be determined by reference to Kabat, E. A. et al, Sequences of Proteins of Immunological Interest. 4th Edition. US Department of Health and Human Services. 1987, and updates thereof, now available on the Internet (http://immuno.bme.nwu.edu)).

The variable domains may be derived from any germline or rearranged human variable domain, or may be a synthetic variable domain based on consensus sequences of known human variable domains. The CDR-derived sequences of the invention, as defined in the preceding paragraph, may be introduced into a repertoire of variable domains lacking CDR regions, using recombinant DNA technology.

For example, Marks et al (Bio/Technology, 1992, 10:779-783) describe methods of producing repertoires of antibody variable domains in which consensus primers directed at or adjacent to the 5′ end of the variable domain area are used in conjunction with consensus primers to the third framework region of human VH genes to provide a repertoire of VH variable domains lacking a CDR/CDRs. Marks et al further describe how this repertoire may be combined with a CDR of a particular antibody. The repertoire may then be displayed in a suitable host system such as the phage display system of WO92/01047 so that suitable specific binding members may be selected. A repertoire may consist of from anything from 10⁴ individual members upwards, for example from 10⁶ to 10⁸ or 10¹⁰ members. Analogous shuffling or combinatorial techniques are also disclosed by Stemmer (Nature, 1994, 370:389-391), who describes the technique in relation to a β-lactamase gene but observes that the approach may be used for the generation of antibodies.

A further alternative is to generate novel VH or VL regions carrying the CDR-derived sequences of the invention using random mutagenesis of, for example, the Ab VH or VL genes to generate mutations within the entire variable domain. Such a technique is described by Gram et al (1992, Proc. Natl. Acad. Sci., USA, 89:3576-3580), who used error-prone PCR. Another method which may be used is to direct mutagenesis to CDR regions of VH or VL genes. Such techniques are disclosed by Barbas et al, (1994, Proc. Natl. Acad. Sci., USA, 91:3809-3813) and Schier et al (1996, J. Mol. Biol. 263:551-567).

All the above described techniques are known as such in the art and in themselves do not form part of the present invention. The skilled person will be able to use such techniques to provide specific binding members of the invention using routine methodology in the art.

A substantial portion of an immunoglobulin variable domain will comprise at least the three CDR regions, together with their intervening framework regions. Preferably, the portion will also include at least about 50% of either or both of the first and fourth framework regions, the 50% being the C-terminal 50% of the first framework region and the N-terminal 50% of the fourth framework region. Additional residues at the N-terminal or C-terminal end of the substantial part of the variable domain may be those not normally associated with naturally occurring variable domain regions. For example, construction of specific binding members of the present invention made by recombinant DNA techniques may result in the introduction of N- or C-terminal residues encoded by linkers introduced to facilitate cloning or other manipulation steps. Other manipulation steps include the introduction of linkers to join variable domains of the invention to further protein sequences including immunoglobulin heavy chains, other variable domains (for example in the production of diabodies) or protein labels as provided herein and/or known to those of skill in the art.

Although in a preferred aspect of the invention specific binding members comprising a pair of binding domains based on sequences substantially set out in FIGS. 12, 13 and/or 14 are preferred, single binding domains based on either of these sequences form further aspects of the invention. In the case of the binding domains based on the sequence substantially set out in FIGS. 12, 13 and/or 14 or in Table 1, such binding domains may be used as targeting agents for HLA-B27 homodimers in a mammal or on cells, particularly immune system cells, particularly monocytes, since it is known that immunoglobulin VH domains are capable of binding target antigens in a specific manner.

This may be achieved by phage display screening methods using the so-called hierarchical dual combinatorial approach as disclosed in U.S. Pat. No. 5,969,108 in which an individual colony containing either an H or L chain clone is used to infect a complete library of clones encoding the other chain (L or H) and the resulting two-chain specific binding member is selected in accordance with phage display techniques such as those described in that reference. This technique is also disclosed in Marks et al, ibid. Phage library and phage display selection systems and techniques are also provided herein.

Specific binding members of the present invention may further comprise antibody constant regions or parts thereof. For example, specific binding members based on the sequences of FIGS. 12, 13 and 14 may be attached at their C-terminal end to antibody light chain constant domains including human Cκ or Cλ chains, preferably Cλ chains. Similarly, specific binding members based on the sequences of FIG. 12, 13 or 14 may be attached at their C-terminal end to all or part of an immunoglobulin heavy chain derived from any antibody isotype, e.g. IgG, IgA, IgE, IgD and IgM and any of the isotype sub-classes, particularly IgG1, IgG2b, and IgG4. IgG1 is preferred.

The antibodies, or any fragments thereof, may be conjugated or recombinantly fused to any cellular toxin, bacterial or other, e.g. pseudomonas exotoxin, ricin, or diphtheria toxin. The part of the toxin used can be the whole toxin, or any particular domain of the toxin. Such antibody-toxin molecules have successfully been used for targeting and therapy of different kinds of cancers, see e.g. Pastan, Biochim Biophys Acta. 1997 Oct. 24; 1333(2):C1-6; Kreitman et al., N Engl J Med. 2001 Jul. 26; 345(4):241-7; Schnell et al., Leukemia. 2000 January; 14(1):129-35; Ghetie et al., Mol Biotechnol. 2001 July; 18(3): 251-68.

Bi- and tri-specific multimers can be formed by association of different scFv molecules and have been designed as cross-linking reagents for T-cell recruitment into tumors (immunotherapy), viral retargeting (gene therapy) and as red blood cell agglutination reagents (immunodiagnostics), see e.g. Todorovska et al., J Immunol Methods. 2001 Feb. 1; 248(1-2):47-66; Tomlinson et al., Methods Enzymol. 2000; 326:461-79; McCall et al., J Immunol. 2001 May 15; 166(10):6112-7.

Fully human antibodies can be prepared by immunizing transgenic mice carrying large portions of the human immunoglobulin heavy and light chains. These mice, examples of such mice are the Xenomouse™ (Abgenix, Inc.) (U.S. Pat. Nos. 6,075,181 and 6,150,584), the HuMAb-Mouse™ (Medarex, Inc./GenPharm) (U.S. Pat. Nos. 5,545,806 and 5569825), the TransChromo Mouse™ (Kirin) and the KM Mouse™ (Medarex/Kirin), are well known within the art. Antibodies can then be prepared by, e.g. standard hybridoma technique or by phage display. These antibodies will then contain only fully human amino acid sequences. Fully human antibodies can also be generated using phage display from human libraries. Phage display may be performed using methods well known to the skilled artisan, and as provided herein as in Hoogenboom et al and Marks et al (Hoogenboom H R and Winter G. (1992) J Mol Biol. 227(2):381-8; Marks J D et al (1991) J Mol Biol. 222(3):581-97; and also U.S. Pat. Nos. 5,885,793 and 5,969,108).

Antibodies of the invention may be labelled with a detectable or functional label. Detectable labels include, but are not limited to, radiolabels such as the isotopes ³H, ¹⁴C, ³²P, ³⁵S, ³⁶Cl, ⁵¹Cr, ⁵⁷Co, ⁵⁸Co, ⁵⁹Fe, ⁹⁰Y, ¹²¹I, ¹²⁴I, ¹²⁵I, ¹³¹I, ¹¹¹In, ¹¹⁷Lu, ²¹¹At, ¹⁹⁸Au, ⁶⁷Cu, ²²⁵Ac, ²¹³Bi, ⁹⁹Tc and ¹⁸⁶Re, which may be attached to antibodies of the invention using conventional chemistry known in the art of antibody imaging. Labels also include fluorescent labels (for example fluorescein, rhodamine, Texas Red) and labels used conventionally in the art for MRI-CT imaging. They also include enzyme labels such as horseradish peroxidase, β-glucoronidase, β-galactosidase, urease. Labels further include chemical moieties such as biotin which may be detected via binding to a specific cognate detectable moiety, e.g. labelled avidin. Functional labels include substances which are designed to be targeted to the site of a tumor to cause destruction of tumor tissue. Such functional labels include cytotoxic drugs such as 5-fluorouracil or ricin and enzymes such as bacterial carboxypeptidase or nitroreductase, which are capable of converting prodrugs into active drugs at the site of a tumor.

Also, antibodies including fragments thereof, and drugs that modulate the production or activity of the specific binding members, antibodies and/or their subunits may possess certain diagnostic applications and may for example, be utilized for the purpose of detecting and/or measuring conditions such as arthritis, spondyloarthritides, AS, reA, conditions related to or resulting from hyperproliferative cell growth or the like. For example, the specific binding members, antibodies or their subunits may be used to produce both polyclonal and monoclonal antibodies to themselves in a variety of cellular media, by known techniques such as the hybridoma technique utilizing, for example, fused mouse spleen lymphocytes and myeloma cells. Likewise, small molecules that mimic or antagonize the activity(ies) of the specific binding members of the invention may be discovered or synthesized, and may be used in diagnostic and/or therapeutic protocols.

The radiolabelled specific binding members, particularly antibodies and fragments thereof, are useful in in vitro diagnostics techniques and in in vivo radioimaging techniques and in radioimmunotherapy. In the instance of in vivo imaging, the specific binding members of the present invention may be conjugated to an imaging agent rather than a radioisotope(s), including but not limited to a magnetic resonance image enhancing agent, wherein for instance an antibody molecule is loaded with a large number of paramagnetic ions through chelating groups. Examples of chelating groups include EDTA, porphyrins, polyamines crown ethers and polyoximes. Examples of paramagnetic ions include gadolinium, iron, manganese, rhenium, europium, lanthanium, holmium and ferbium. In a further aspect of the invention, radiolabelled specific binding members, particularly antibodies and fragments thereof, particularly radioimmunoconjugates, are useful in radioimmunotherapy, particularly as radiolabelled antibodies for cell therapy. In a still further aspect, the radiolabelled specific binding members, particularly antibodies and fragments thereof, are useful in radioimmuno-guided surgery techniques, wherein they can identify and indicate the presence and/or location of HLA-B27 homodimers, HLA-B27 homodimer expressing cells, hyperproliferative cells, prior to, during or following procedures to remove or reduce such cells.

Immunoconjugates or antibody fusion proteins of the present invention, wherein the specific binding members, particularly antibodies and fragments thereof, of the present invention are conjugated or attached to other molecules or agents further include, but are not limited to binding members conjugated to a chemical ablation agent, toxin, immunomodulator, cytokine, cytotoxic agent, chemotherapeutic agent or drug.

Radioimmunotherapy (RAIT) has entered the clinic and demonstrated efficacy using various antibody immunoconjugates. ¹³¹I labeled humanized anti-carcinoembryonic antigen (anti-CEA) antibody hMN-14 has been evaluated in colorectal cancer (Behr T M et al (2002) Cancer 94(4 Suppl):1373-81) and the same antibody with ⁹⁰Y label has been assessed in medullary thyroid carcinoma (Stein R et al (2002) Cancer 94(1):51-61). Radioimmunotherapy using monoclonal antibodies has also been assessed and reported for non-Hodgkin's lymphoma and pancreatic cancer (Goldenberg D M (2001) Crit Rev Oncol Hematol 39(1-2):195-201; Gold D V et al (2001) Crit Rev Oncol Hematol 39 (1-2) 147-54). Radioimmunotherapy methods with particular antibodies are also described in U.S. Pat. Nos. 6,306,393 and 6,331,175. Radioimmunoguided surgery (RIGS) has also entered the clinic and demonstrated efficacy and usefulness, including using anti-CEA antibodies and antibodies directed against tumor-associated antigens (Kim J C et al (2002) Int J Cancer 97(4):542-7; Schneebaum S et al (2001) World J Surg 25(12):1495-8; Avital S et al (2000) Cancer 89(8):1692-8; McIntosh D G et al (1997) Cancer Biother Radiopharm 12 (4):287-94).

In vivo animal models of Spondyloarthritides (SpA) conditions, AS, reA may be utilized by the skilled artisan to further or additionally screen, assess, and/or verify the specific binding members and antibodies or fragments thereof of the present invention, including further assessing HLA-B27 homodimer modulation and inhibiting SpA conditions in vivo and inhibiting arthritis or inflammation. Such animal models include, but are not limited to models of osteoarthritis, rheumatoid arthritis. Particular models include transgenic rodent models of spondylarthritis, HLA-B27 transgenic animals, HLA-B*2705/human β2m transgenic mice.

Antibodies of the present invention may be administered to a mammal or patient in need of treatment via any suitable route, including by injection intramuscularly, into the bloodstream, into the spine, or directly into a site affected by the SpA condition. The precise dose will depend upon a number of factors, including whether the antibody is for diagnosis or for treatment, the size and location of the tumor, the precise nature of the antibody (whether whole antibody, fragment, diabody, etc), and the nature of the detectable or functional label attached to the antibody. Where a radionuclide is used for therapy, a suitable maximum single dose may be about 45 mCi/m², to a maximum of about 250 mCi/m². Preferable dosage is in the range of 15 to 40 mCi, with a further preferred dosage range of 20 to 30 mCi, or 10 to 30 mCi. Such therapy may require bone marrow or stem cell replacement. A typical antibody dose for either tumor imaging or tumor treatment will be in the range of from 0.5 to 40 mg, preferably from 1 to 4 mg of antibody in F(ab′)2 form. Naked antibodies are preferably administered in doses of 20 to 1000 mg protein per dose, or 20 to 500 mg protein per dose, or 20 to 100 mg protein per dose. This is a dose for a single treatment of an adult patient, which may be proportionally adjusted for children and infants, and also adjusted for other antibody formats, in proportion for example to molecular weight. Treatments may be repeated at daily, twice-weekly, weekly or monthly intervals, at the discretion of the physician.

### Pharmaceutical and Therapeutic Compositions

Specific binding members of the present invention will usually be administered in the form of a pharmaceutical composition, which may comprise at least one component in addition to the specific binding member. Thus pharmaceutical compositions according to the present invention, and for use in accordance with the present invention, may comprise, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. intravenous, or by deposition at a tumor site.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

A composition may be administered alone or in combination with other treatments, therapeutics or agents, either simultaneously or sequentially dependent upon the condition to be treated. In addition, the present invention contemplates and includes compositions comprising the binding member, particularly antibody or fragment thereof, herein described and other agents or therapeutics such as anti-inflammatory agents, antibodies, or immune modulators. Other treatments or therapeutics may include the administration of suitable doses of pain relief drugs such as non-steroidal anti-inflammatory drugs (e.g. aspirin, paracetamol, ibuprofen or ketoprofen) or opiates such as morphine, or anti-emetics. Thus, these agents may be specific anti-inflammatory agents, or immune cell response modulators or may be more general agents such as NSAIDs, steroids. In addition, the composition may be administered with hormones such as dexamethasone, immune modulators, such as interleukins, tumor necrosis factor (TNF) or other growth factors, colony stimulating factors, or cytokines which stimulate the immune response and reduction or elimination of cancer cells or tumors. The composition may also be administered with, or may include combinations along with other anti-HLA antigen antibodies.

In addition, the present invention contemplates and includes therapeutic compositions for the use of the binding member in combination with conventional radiotherapy.

The present invention further contemplates therapeutic compositions useful in practicing the therapeutic methods of this invention. A subject therapeutic composition includes, in admixture, a pharmaceutically acceptable excipient (carrier) and one or more of a specific binding member, polypeptide analog thereof or fragment thereof, as described herein as an active ingredient. In a preferred embodiment, the composition comprises an antigen capable of modulating the specific binding of the present binding member/antibody with a target cell.

The preparation of therapeutic compositions which contain polypeptides, analogs or active fragments as active ingredients is well understood in the art. Typically, such compositions are prepared as injectables, either as liquid solutions or suspensions. However, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A polypeptide, analog or active fragment can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic antibody- or active fragment-containing compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of HLA-B27 homodimer binding capacity desired. Precise amounts of active ingredient required to be administered depend on the judgment of the practitioner and are peculiar to each individual. Suitable regimes for initial administration and follow on administration are also variable, and may include an initial administration followed by repeated doses at one or more hour intervals by a subsequent injection or other administration. Alternatively, continuous intravenous infusion sufficient to maintain appropriate and sufficient concentrations in the blood or at the site of desired therapy are contemplated.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Diagnostic Assays

The present invention also relates to a variety of diagnostic applications, including methods for detecting the expression of or elevated presence of B27₂, HLA-B27 homodimers, HLA-B27-mediated mediated disease or conditions, SpA conditions, reactive arthritis, Ankylosing Spondylitis, or more inflammatory or arthritic conditions, by reference to their ability to be recognized by the present specific binding member(s). Peptide complexes can be identified, targeted, labeled, and/or quantitated on stromal cells, fibroblast cells and/or tumor cells.

Diagnostic applications of the specific binding members of the present invention, particularly antibodies and fragments thereof, include in vitro and in vivo applications well known and standard to the skilled artisan and based on the present description. Diagnostic assays and kits for in vitro assessment and evaluation of tumor and cancer status, may be utilized to diagnose, evaluate and monitor patient samples including those known to have or suspected of having AS, reA, a Spondyloarthritic condition, a condition related to hyperproliferative cell growth or an arthritic condition. The assessment and evaluation of HLA-B27 disease or SpA condition status is also useful in determining the suitability of a patient for a clinical trial of a drug or for the administration of a particular chemotherapeutic agent or specific binding member, particularly an antibody, of the present invention, including combinations thereof, versus a different agent or binding member. This type of diagnostic monitoring and assessment is already in practice utilizing antibodies against the HER2 protein in breast cancer (Hercep Test, Dako Corporation), where the assay is also used to evaluate patients for antibody therapy using Herceptin.

Preferably, the antibody used in the diagnostic methods of this invention is human antibody. More preferably, the antibody is a single chain antibody or domain antibody. In addition, the antibody molecules used herein can be in the form of Fab, Fab′, F(ab′)₂ or F(v) portions of whole antibody molecules, particularly Fab.

As described in detail above, antibody(ies) to B27₂ can be produced and isolated by standard methods including the phage display techniques and mutagenesis and recombinant techniques.

The presence of HLA-B27 homodimers in a sample, a mammal or on cells can be ascertained by the usual in vitro or in vivo immunological procedures applicable to such determinations. A number of useful procedures are known. The procedures and their application are all familiar to those skilled in the art and accordingly may be utilized within the scope of the present invention. The “competitive” procedure is described in U.S. Pat. Nos. 3,654,090 and 3,850,752. The “sandwich” procedure, is described in U.S. Pat. Nos. RE 31,006 and 4,016,043. Still other procedures are known such as the “double antibody,” or “DASP” procedure.

It is notable that prior methods for assessing HLA-B27, were limited by the unavailability of a homodimer specific antibody. Therefore, the homodimer could not readily and directly be assessed without first removing other forms, specifically isolating the homodimers, or assessing other forms simultaneously. WO99/58557 describes the dimer of the HLA-B27 heavy chain, however, detection thereof or determination of levels thereof cannot be achieved directly and specifically without an HLA-B27 homodimer specific reagent such as an antibody of the present invention. WO2004/029628 describes assay methods comprising incubating soluble HLA heavy chain, β₂ microglobulin and peptides to determine the peptides that bind to HLA Class I molecules. As now recognized, the HLA-B27 homodimers B27₂ do not associate with β2m, and therefore this prior disclosed method is not useful or applicable for HLA-homodimer assessment.

In a further embodiment of this invention, commercial test kits suitable for use by a medical specialist may be prepared to determine the presence or absence of aberrant expression of including but not limited to amplified and/or an mutation, in suspected target cells. In accordance with the testing techniques discussed above, one class of such kits will contain at least the labeled or its binding partner, for instance an antibody specific thereto, and directions, of course, depending upon the method selected, e.g., “competitive,” “sandwich,” “DASD” and the like. The kits may also contain peripheral reagents such as buffers, stabilizers, etc.

Accordingly, a test kit may be prepared for the assessment of HLA-B27 diseases or conditions mediated by HLA-B27 homodimers B27₂ comprising:

(a) a predetermined amount of an antibody capable of specifically binding B27₂;

(b) a means for detecting the binding of said antibody to HLA-B27 homodimers; and

(c) directions for use.

A test kit may be prepared for the demonstration of the presence of a Spondyloarthritic condition, particularly selected from AS, reA, uveitis and sacroileitis comprising:

(a) a predetermined amount of at least one labeled immunochemically reactive component obtained by the direct or indirect attachment of the HLA-B27 homodimer specific antibody or fragment or a specific binding partner thereto, to a detectable label;

(b) other reagents; and

(c) directions for use of said kit.

In accordance with the above, an assay system for screening potential drugs effective to modulate the presence, activity or amount of B27₂ and/or the activity or binding of the antibody of the present invention may be prepared. The antigen peptide or the binding member or antibody may be introduced into a test system, and the prospective drug may also be introduced into the resulting cell culture, and the culture thereafter examined to observe any changes in the activity of the cells, binding of the antibody, or amount and extent of HL-B27 homodimers due either to the addition of the prospective drug alone, or due to the effect of added quantities of the known agent(s).

### Nucleic Acids

The present invention further provides an isolated nucleic acid encoding a specific binding member of the present invention. Nucleic acid includes DNA and RNA. In a preferred aspect, the present invention provides a nucleic acid which codes for a polypeptide of the invention as defined above, including a polypeptide as set out in FIG. 12, 13 or 14 (SEQ ID NO: 2, 7, 12, 17, 22 27) or capable of encoding the CDR regions thereof as set out in FIG. 12, 13 or 14 or in Table 1 (SEQ ID NO: 3-5, 8-10, 13-15, 18-20, 23-25, 28-30). Nucleic acids having or comprising sequences as set out in FIG. 12, 13 or 14 (SEQ ID NO: 1, 6, 11, 16, 21, 26) are provided herein.

The present invention also provides constructs in the form of plasmids, vectors, transcription or expression cassettes which comprise at least one polynucleotide as above. The present invention also provides a recombinant host cell which comprises one or more constructs as above. A nucleic acid encoding any specific binding member as provided itself forms an aspect of the present invention, as does a method of production of the specific binding member which method comprises expression from encoding nucleic acid therefor. Expression may conveniently be achieved by culturing under appropriate conditions recombinant host cells containing the nucleic acid. Following production by expression a specific binding member may be isolated and/or purified using any suitable technique, then used as appropriate.

Specific binding members and encoding nucleic acid molecules and vectors according to the present invention may be provided isolated and/or purified, e.g. from their natural environment, in substantially pure or homogeneous form, or, in the case of nucleic acid, free or substantially free of nucleic acid or genes origin other than the sequence encoding a polypeptide with the required function. Nucleic acid according to the present invention may comprise DNA or RNA and may be wholly or partially synthetic.

Systems for cloning and expression of a polypeptide in a variety of different host cells are well known. Suitable host cells include bacteria, mammalian cells, yeast and baculovirus systems. Mammalian cell lines available in the art for expression of a heterologous polypeptide include Chinese hamster ovary cells, HeLa cells, baby hamster kidney cells, cancer cells, ovarian cancer cells and many others. A common, preferred bacterial host is E. coli. The expression of antibodies and antibody fragments in prokaryotic cells such as E. coli is well established in the art.

Suitable vectors can be chosen or constructed, containing appropriate regulatory sequences, including promoter sequences, terminator sequences, polyadenylation sequences, enhancer sequences, marker genes and other sequences as appropriate. Vectors may be plasmids, viral e.g. ‘phage, or phagemid, as appropriate. For further details see, for example, Molecular Cloning: a Laboratory Manual: 2nd edition, Sambrook et al., 1989, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Short Protocols in Molecular Biology, Second Edition, Ausubel et al. eds., John Wiley & Sons, 1992. The disclosures of Sambrook et al. and Ausubel et al. are incorporated herein by reference.

Thus, a further aspect of the present invention provides a host cell containing nucleic acid as disclosed herein. A still further aspect provides a method comprising introducing such nucleic acid into a host cell. The introduction may employ any available technique. For eukaryotic cells, suitable techniques may include calcium phosphate transfection, DEAE-Dextran, electroporation, liposome-mediated transfection and transduction using retrovirus or other virus, e.g. vaccinia or, for insect cells, baculovirus. For bacterial cells, suitable techniques may include calcium chloride transformation, electroporation and transfection using bacteriophage. The introduction may be followed by causing or allowing expression from the nucleic acid, e.g. by culturing host cells under conditions for expression of the gene. The present invention also provides a method which comprises using a construct as stated above in an expression system in order to express a specific binding member or polypeptide as above.

Another feature of this invention is the expression of the DNA sequences disclosed herein. As is well known in the art, DNA sequences may be expressed by operatively linking them to an expression control sequence in an appropriate expression vector and employing that expression vector to transform an appropriate unicellular host. A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col El, pCR1, pBR322, pMB9 and their derivatives, plasmids such as RP4; phage DNAs, e.g., the numerous derivatives of phage X, e.g., NM989, and other phage DNA, e.g., M13 and filamentous single stranded phage DNA; yeast plasmids such as the 2u plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

Any of a wide variety of expression control sequences—sequences that control the expression of a DNA sequence operatively linked to it—may be used in these vectors to express the DNA sequences of this invention. Such useful expression control sequences include, for example, the early or late promoters of SV40, CMV, vaccinia, polyoma or adenovirus, the lac system, the trp system, the TAC system, the TRC system, the LTR system, the major operator and promoter regions of phage X, the control regions of fd coat protein, the promoter for 3-phosphoglycerate kinase or other glycolytic enzymes, the promoters of acid phosphatase (e.g., Pho5), the promoters of the yeast-mating factors, and other sequences known to control the expression of genes of prokaryotic or eukaryotic cells or their viruses, and various combinations thereof.

A wide variety of unicellular host cells are also useful in expressing the DNA sequences of this invention. These hosts may include well known eukaryotic and prokaryotic hosts, such as strains of E. coli, Pseudomonas, Bacillus, Streptomyces, fungi such as yeasts, and animal cells, such as CHO, YB/20, NSO, SP2/0, R1.1, B-W and L-M cells, African Green Monkey kidney cells (e.g., COS 1, COS 7, BSC1, BSC40, and BMT10), insect cells (e.g., Sf9), and human cells and plant cells in tissue culture.

It will be understood that not all vectors, expression control sequences and hosts will function equally well to express the DNA sequences of this invention. Neither will all hosts function equally well with the same expression system. However, one skilled in the art will be able to select the proper vectors, expression control sequences, and hosts without undue experimentation to accomplish the desired expression without departing from the scope of this invention. In selecting an expression control sequence, a variety of factors will normally be considered. These include, for example, the relative strength of the system, its controllability, and its compatibility with the particular DNA sequence or gene to be expressed, particularly as regards potential secondary structures. Suitable unicellular hosts will be selected by consideration of, e.g., their compatibility with the chosen vector, their secretion characteristics, their ability to fold proteins correctly, and their fermentation requirements, as well as the toxicity to the host of the product encoded by the DNA sequences to be expressed, and the ease of purification of the expression products. Considering these and other factors a person skilled in the art will be able to construct a variety of vector/expression control sequence/host combinations that will express the DNA sequences of this invention on fermentation or in large scale animal culture.

As mentioned above, a DNA sequence encoding a specific binding member can be prepared synthetically rather than cloned. The DNA sequence can be designed with the appropriate codons for the specific binding member amino acid sequence. In general, one will select preferred codons for the intended host if the sequence will be used for expression. The complete sequence is assembled from overlapping oligonucleotides prepared by standard methods and assembled into a complete coding sequence. See, e.g., Edge, Nature, 292:756 (1981); Nambair et al., Science, 223:1299 (1984); Jay et al., J. Biol. Chem., 259:6311 (1984). Synthetic DNA sequences allow convenient construction of genes which will express specific binding member analogs or “muteins”. Alternatively, DNA encoding muteins can be made by site-directed mutagenesis of native specific binding member genes or cDNAs, and muteins can be made directly using conventional polypeptide synthesis.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### Example 1

Ankylosing Spondylitis (AS) is strongly correlated with possession of the HLA Class I allele B27. HLA-B27 forms heterotrimers (B27) with beta 2-microglobulin (β2m) and peptide, but, unusually, also forms abnormal β2 microglobulin-free heavy chain homodimers (B27₂). B27₂ bind Killer Immunoglobulin-like Receptors (KIR) and Leukocyte Immunoglobulin-like Receptors (LILR) that are expressed on NK cells, T cells and myeloid cells. However, the extent of B27₂ cell surface expression in AS patients and the functional consequences of its receptor interactions in AS pathogenesis are not known. To address this issue, we generated an antibody (HD6) that specifically recognized B27₂ but not B27 or any other HLA heterotrimer complexes. Using HD6 antibody, we have demonstrated for the first time that in HLA-B27+ AS patients, B27₂ expression was significantly increased on blood and synovial fluid derived monocytes. Furthermore, in healthy individuals, possession of the HLA-B27 allele correlated with low-level expression of B27₂ on B-lymphocytes but not on other lymphocytes. HD6 inhibited binding of B27₂ to KIR3DL1, KIR3DL2 and LILRB2 immunoreceptors and B27₂-mediated survival and proliferation of human NK cells expressing KIR3DL2.

Introduction

Possession of the Human Leukocyte Antigen (HLA) class I B27 molecule is strongly associated with AS and other Spondyloarthritides (SpA)(1-3). Despite extensive investigations, the understanding of the pathogenic role of HLA-B27 in disease is limited (4). Under normal physiological conditions, HLA-B27 heavy chains form heterotrimeric complexes in the endoplasmic reticulum together with β2 microglobulin (β2m) and intracellular peptides, derived from self-proteins, viruses and bacteria. These heterotrimeric complexes (henceforth called HLA-B27) egress to the cell surface where they are recognized by CD8+ cytotoxic T cells through their respective T cell receptors (5). However, B27 heavy chains can also form β2m-free disulfide-bonded heavy chain homodimers (6) (FIG. 6A). These homodimers, which we have termed B27₂, assemble both intracellularly during maturation (7-9) and are expressed at the cell surface following endosomal recycling of heterotrimers (10). The ability of B27 to form disulphide bonds through its unpaired cysteine at position 67 is both highly unusual and critical for cell surface homodimer expression (10), although certain other murine and human class I alleles can form homodimers through alternative cysteines (11;12).

The “homodimer” theory of AS pathogenesis suggests that inflammation results from the interaction of aberrant cell-surface-expressed B27₂ with immune cells bearing innate immune receptors (14). B27₂ binds to several Killer Immunoglobulin-like Receptors (KIR) and related Leukocyte Immunoglobulin-like Receptors (LILR), including KIR3DL1, KIR3DL2 and LILRB2 (13-15). The binding pattern is different to that of heterotrimeric HLA-B27 complexes, which do not bind KIR3DL2 (14). Although rodents do not possess KIRs, rodent Paired Immunoglobulin Receptors (PIR) are ligands for B27₂(16). In the HLA-B27 transgenic rat AS model, disease is dependent on high B27 copy number, can be transferred by bone-marrow derived cells (17), but does not require CD8+ T cells (18). The latter data strongly suggest that classical recognition of heterotrimeric complexes by CD8-dependent T cells is not critical for pathogenesis. The occurrence of related disease in HLA-B27-transgenic β2m-deficient mice, and disease amelioration using the human class I heavy chain-specific HC10 antibody, provides evidence for a direct role for B27 heavy chains (19).

Firm support for the theory that cell surface B27₂ contribute to AS/SpA pathogenesis has been lacking due to the lack of a B27₂-specific antibody. The HC10 mAb binds B27₂ but also recognizes other B27 heavy chain structures (including free heavy chains and multimers) and also binds to HLA-B, C and some A alleles (20;21). Thus, HC10 is not a specific reagent for B27₂ or indeed for B27 heavy chains. Although increased HC10 expression on peripheral blood monocytes of AS patients has been demonstrated (22;23), the exact nature of the molecules recognized is unknown. Hence, there is a pressing need to develop new reagents to detect B27 homodimer expression on human cells, and to determine if B27₂ have functional effects on immune receptor recognition that contribute to AS/SpA pathogenesis. Further, the availability of a B27 homodimer specific antibody provides opportunity to specifically ameliorate B27 homodimer mediated or associated disease and pathologies.

We aimed to establish and characterize novel antibodies from a phage display library that would specifically recognize B27₂ in order to better understand the homodimer role in AS/SpA pathogenesis.

Materials and Methods

Cell Lines, Plasmids and Patient Samples

A glutamine synthetase gene selection system was used (LONZA Biologics, Basel, Switzerland) consisting of mouse myeloma cells (NSO cells) and a mouse/human chimeric IgG1 expressing vector pEE12.4. Cells were grown in 10% RPMI without glutamine, supplemented with penicillin, streptomycin and glutamate (GIBCO, Karlsruhe, Germany). LBL721.220 transfectants including 0.220B27, Baf3 KIR3DL1+, KIR3DL2+, were generated as described previously (13). NK-YT cells expressing KIR3DL2+ and Baf3 cells expressing LILRB2 were generated by lentiviral transduction. 10 ml peripheral blood samples were obtained from patients with AS/SpA and Rheumatoid Arthritis attending the Nuffield Orthopaedic Centre, and from healthy controls, with informed consent and appropriate ethical permission (COREC 06/Q1606/139).

Monoclonal Antibodies

KIR3DL1− (DX9, mouse IgG1) and KIR3DL2-specific (DX31, mouse IgG2a) mAbs were a gift from Jo Phillips (DNAX Palo Alto, Calif.). HC10 (IgG2a), which recognizes β2m-free class I heavy chains was a gift from Dr. Hidde Ploegh (MIT, MA). W6/32 (IgG2a, Dako, UK) recognizes human HLA class I heavy chains associated with β2m. ME1 recognizes HLA-B27, B7, B42, B67, B73, and Bw22. Antibodies were purified from hybridoma supernatants by protein-A sepharose. Rat anti-HA tag antibody (clone 3F10, Roche, UK) was used for Western Blotting. Isotype control mAb mouse IgG2a or IgG 1 were purchased (Becton Dickinson, UK).

Preparation of B27 Homodimer and Other HLA Complexes

HLA-B*2705 homodimer (two heavy chains with cysteine 67 disulphide bond) and heterotrimer (heavy chain, β2m & peptide) complexes were prepared as described previously (14). Briefly, recombinant HLA-B27 was expressed in E. Coli recA-BL21 (DE3) pLysS (GOLD) (Stratagene, UK), purified on Ni-NTA resin (Fast-Flow; Amersham Pharmacia Biotech, Little Chalfont, UK) and refolded by limiting dilution with or without β2m in the presence of Influenza Nucleoprotein NP383-391 peptide epitope SRYWAIRTR or EBV EBNA3C epitope RRIYDLIEL (14). Monomeric and dimeric forms were purified by FPLC purification and confirmed by non-reducing and reducing SDS-PAGE. After biotinylation, phycoerythrin (PE) labeled extravidin (Sigma, Poole, UK) was used to prepare tetramer complexes. Control HLA heterotrimeric complexes were refolded with the following peptide epitopes: HLA-A*0201 (“HLA-A2”) with SLYNTVATL (SEQ ID NO: 31), HLA-A*0301 with RLRAEAQVK (SEQ ID NO: 32), HLA-A*2401 with RYPLTFGW (SEQ ID NO: 33), or HLA-B*0702 (“HLA-B7”) with LPFDKTUM (SEQ ID NO: 34) or RPMTYKAAL (SEQ ID NO: 35) as described (13).

Selection of Dimer Specific Fab Antibodies by Phage Display

Human Fab antibodies were selected from a fully human Fab antibody library (kindly provided by Dyax, MA, USA) as described previously (24). Briefly, dynabeads M-280 streptavidin (Dynal, Oslo, Norway) and phage particles were blocked with 2% nonfat dry milk powder (ROTH, Switzerland) in PBS at room temperature (RT). Then, biotinylated B27₂ molecules and phage particles were incubated with streptavidin dynabeads for 1 h. After multiple washes with 0.3% Tween20-PBS, specific binders (phages) were eluted with 100 mM triethanolamine (pH-10.5) for 5 min. and neutralized with 0.5 ml of 1M Tris-HCl, pH-7.4. Three positive selection rounds were performed with decreasing concentrations (250, 125 and 50 nM) of B27₂ at RT. A negative selection step was performed by pre-incubating the amplified phages from round 1 with B27 heterotrimer complex prior to selection round 2 on B27₂. Specific phage binders from each round were rescued by infecting E. Coli strain TG 1 (Zymo Research, Switzerland) grown in 2YT broth (BD/DIFCO, Switzerland), supplemented with 100 μg/ml ampicillin and 2% glucose (2YT-AG). Rescued clones from selection round 3 were cultured on 2YT-AG agar plates overnight at 30° C. Individual TG1 colonies were infected with helper phage M13K07 (NEB, Switzerland) at 1:20 ratio and grown in 2YT broth supplemented with ampicillin and 25 μg/ml of kanamycin (2YT-AK) over night (O/N) at 30° C. Supernatants were tested for binders in ELISA. Diversity of the selected binders was determined by colony PCR (Red-Taq readymix, Sigma, Switzerland), finger printing (BstNI digestion) and confirmed by DNA sequencing with heavy and light chain specific primers.

Expression and Purification of Fab Fragments

Fabs were expressed and purified as described previously (25). Briefly, TG1 cultures were grown at 37° C. until the OD reached 0.8-1.0 at A600 nm in 2YT supplemented with ampicillin and 0.1% glucose. Cultures were induced with 1 mM IPTG (Roche, Switzerland) and further incubated at 30° C. for 4 h. Periplasmic portions were isolated using 2M sucrose and Fab molecules were purified with Talon resin (BD Clontech, Switzerland). Purity and molecular weight were confirmed by SDS-PAGE.

Generation of Chimeric IgGs

Specific human Fab binders were converted into chimeric IgG molecules exhibiting mouse Fc using a GS (Glutamine Synthetase) gene expression system (LONZA, Switzerland). Briefly, the variable regions of heavy chain and light chain were amplified with DraIII or RsrII primers. Variable chain amplicons of approximately 400 by were digested with respective enzymes and were cloned into the pEE-12.4 vector possessing mouse Fc and human kappa light chain. 40 μg of plasmid DNA was linearized with Pvu-I and transfected into NSO cells by electroporation at 250V and 400 μF with 6-7.5 millisecond time constant (Genepulser II, BioRad, Switzerland). Clones were grown in glutamine free DMEM (GIBCO, Germany) supplemented with 10% FBS, penicillin, streptomycin and GS supplement (Invitrogen, Switzerland). After electroporation, cells were grown in DMEM containing 7.5 μM of MSX (Methionine Sulfoximine—a glutamine synthetase inhibitor, sigma, Switzerland) supplemented with glutamine. Secreted IgGs were purified with Capture Select Fab kappa affinity matrix (BAC, Netherlands). Purity and molecular weights were determined by SDS-PAGE.

ELISA with Phages, Fabs and HD6 IgG

Enzyme Linked Immuno Sorbent Assays (ELISA) were performed using plate-bound HLA complexes. Maxisorp (Nunc, Switzerland) 96 well plates were coated with 2 μg/ml biotinylated BSA, streptavidin (10 μg/ml) (Promega, Switzerland) at 37° C. for 30 minutes each. Biotinylated HLA complexes were incubated for 1 h and wells were blocked with 5% Milk powder-PBS solution. Phages, Fabs or IgGs were allowed 1 h for binding. Following three washes with 0.3% PBS-T, anti-M13 (GE/Amersham, Switzerland) or anti-human Fab (Sigma, Switzerland) or anti-mouse Fc (BioRad, Switzerland), HRP-conjugated antibodies were used as detector. After three washes, tetra-methylbenzidine was used as substrate for color development. 2N H₂SO₄ was used to stop the reaction and absorbance was read at 450 nm (Wallac Victor 2, Perkin-Elmer, Switzerland).

Surface Plasmon Resonance

Surface Plasmon Resonance (SPR) measurements were performed using a Biacore 3000. 240 response units (RU) of biotinylated B27₂ were immobilized on a streptavidin-coated chip (in flow cell 2). Fab6 was injected over flow cells 1 and 2 at increasing concentrations from 0 μg/ml to 200 μg/ml. The flow cell was regenerated by injecting glycine pH 2.5 after each Fab injection. Experiments were performed three times at 25° C. and Kd values were obtained after subtraction of background from flow cell 1.

Competition ELISA

5 μg/ml of either HD6 or HC10 antibodies were coated on Nunc maxisorp plates (1 h at RT). After blocking the wells with 5% milk powder in PBS, 1 μg/ml of B27₂ and 10 μg/ml competing antibody was incubated in triplicates (1 h at RT). Streptavidin HRP (Roche, Switzerland) was incubated at 1:2,000 dilution for an additional hour. Upon stringent washes with 0.1% Tween20-PBS, wells were developed as described above in ELISA method.

Flow Cytometry

LBL721.220 cells were stained (200,000 cells/500) with 1 μg of HD6, HC10, ME1 or W6/32, respectively on ice for 20 mins. Goat anti-mouse IgG conjugated to PE was used as detector antibody. Cells were washed 3 times in 0.1% FBS-PBS and fixed with 2% paraformaldehyde. HLA tetramer flow cytometry was conducted using freshly prepared extravidin PE-labelled homodimer or heterotrimer tetramers, with or without pre-incubation of 1 μg HD6 or HC10 (20 min. on ice). Quantification of cell surface HD6-reactive molecules was carried out using Quantibrite beads (Becton Dickinson, Switzerland) according to the manufacturer instructions. Cytometric analysis was performed on a CyAn ADP and data were analyzed using FloJo software (Version 7.2.5, Tree Star, UK).

NK Cell Survival and Proliferation Assays

100,000 lentivirally transduced KIR3DL2 hYT NK were labelled with CFSE according to the manufacturer's instructions (Invitrogen, UK), and incubated with irradiated LBL.721.220 cells transfected with HLA-B27 or control HLA for 3 days in RPMI 1640 supplemented with 10% fetal calf serum, penicillin, streptomycin and L-glutamine. Subsequently cells were stained with AnnexinV APC (BD Bioscience, UK) and pacific blue stain (Live Dead) for FACS analysis. Proliferation of viable CFSE-labelled cells was analysed after gating out cells staining positive for pacific blue and AnnexinV. Total viable CFSE+ cell numbers were calculated from the number of flow-count fluorospheres (Beckman Coulter, UK) at 100,000 beads/ml counted per sample. For blocking experiments, LBL.721.220 HLA-B27 transfectants were first stained with HD6 or IgG1 isotype control mAb (10 μg/ml) for 20 mins on ice, followed by addition of transduced or control hYT NK cells for 3 days as described previously.

IFNγ ELISA

IFN gamma production was performed with minor changes as described (13). Briefly, 50,000 KIR3DL2-expressing NK cells (HyT) in 100 ul were co-cultured with an equal number and volume of 0.220 cells expressing B7, B27 and B27₂. 10 ug of either HD6 or HC10 antibodies were pre-incubated with antigen presenting cells (0.220) on ice for 20 mins. Cells were allowed for IFNγ production at 37° C./5% CO2 for 12 hours. Supernatants were collected after pelleting the cells, and 1:2 diluted supernatants were used in IFNγ ELISA (Roche, UK) according to the manufacturer's instructions.

Statistical Analysis

Student's unpaired t-test was used to determine p-values between two groups and a p-value of <0.05 was considered as significant. Non-linear fit and binding saturations in affinity ELISA were conducted using Graphpad prism software. A Langmuir 1:1 binding fit measurements from SPR were calculated using Bio-Evaluation software.

Results

Selection of B27₂ Specific Antibodies

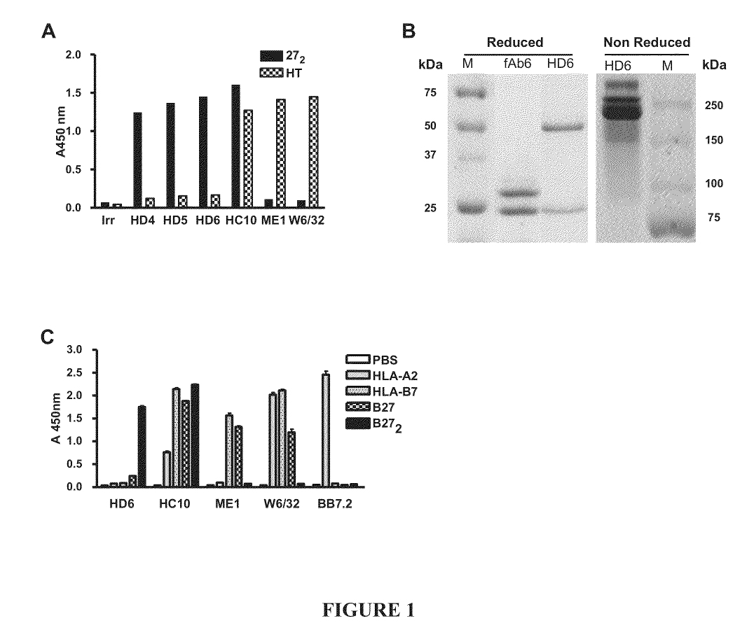

Biotinylated recombinant B27₂ was used for positive selection and heterotrimeric HLA-B27 for negative selection of Fab antibodies from a phage library (FIG. 6A) (24;25). Twelve different antibodies were isolated and further characterized by colony PCR, finger printing and sequence analysis. Three promising candidate Fabs (clones 4-6, denoted HD4, HD5 and HD6) were converted into chimeric antibodies comprising human Fab₂ with murine IgG1 Fc and showed specificity for B27₂ complexes in ELISA (FIG. 1A). One of these IgG antibodies, HD6, was selected for further characterization and a stable mammalian cell line generated for high-level IgG production (FIG. 1B). The integrity and chimeric nature of the recombinant HD6 IgG antibody was confirmed by sandwich ELISA (FIG. 6B).

HD6 Binds to Recombinant B27₂ and Differs in Specificity from HC10

We next compared the specificity of HD6 with a panel of antibodies recognizing HLA-Class I molecules, including HC10, known to bind β2m-free heavy chains (20). HD6 bound to recombinant B27₂ complexes but not to HLA-A2, B7 or B27 complexes in ELISA (FIG. 1C). HC10 also recognized B27₂ as described previously (6) but, additionally, bound to other complexes including HLA-B7 and, to a lesser extent, HLA-A2. By contrast, the heterotrimer Bw4-specific antibody ME1 recognized only heterotrimeric HLA-B7 and HLA-B27 but not B27₂. Treatment with the reducing agent DTT abrogated recognition of B27₂ by HD6 but not by HC10 (FIG. 7A and data not shown), suggesting an important role for the disulphide bond for epitope recognition by HD6. In ELISA competition experiments, HD6 binding to B27₂ was not inhibited by excess of HC10 antibody and vice versa (FIG. 7B) suggesting that HD6 and HC10 recognize different B27₂ epitopes. However, in order to formally exclude the possibility that these results were due to differences in binding affinity, we determined the affinities of HD6 and HC10 for B27₂ using both Surface Plasmon Resonance (SPR) and ELISA. Fab fragments of HD6 and HC10 gave dissociation constants of 270 nM (HD6) and 220 nM (HC10) in a Langmuir 1:1 fit model by SPR (FIG. 8A). In ELISA using intact IgG antibodies, non-linear fit binding saturation was observed for HD6 at 1.77 nM and 1.0 nM for HC10 (FIG. 8B). Thus, HD6 has comparable affinity to HC10 for B27₂ with a markedly different binding specificity.

Recognition of Cell Surface Homodimers (B27₂) by HD6 Antibody

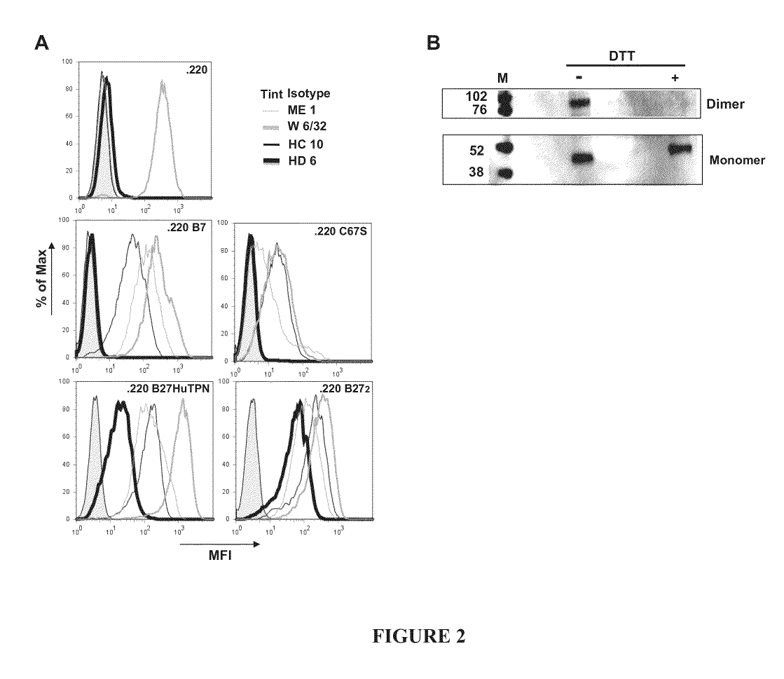

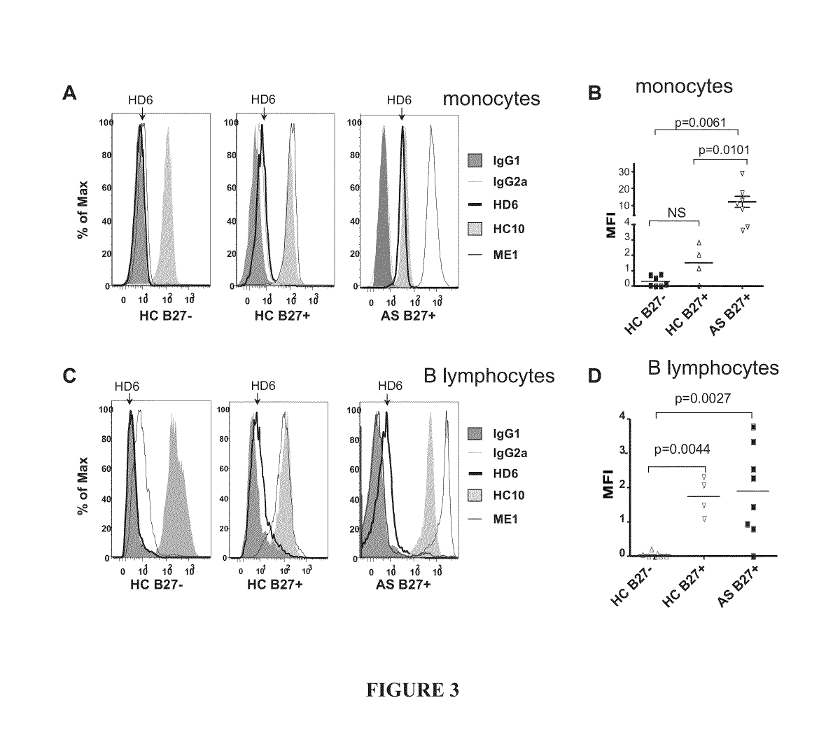

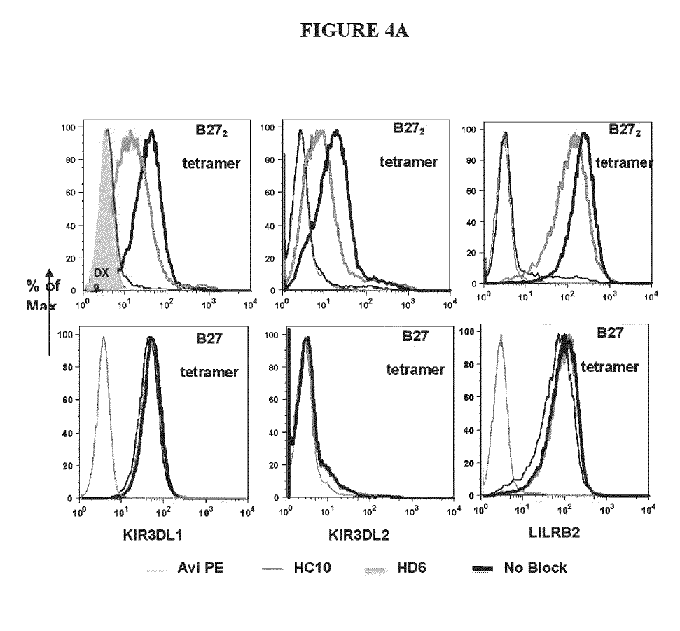

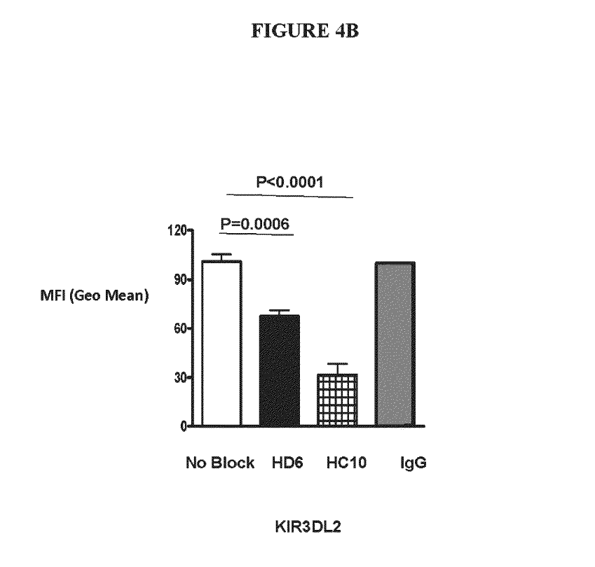

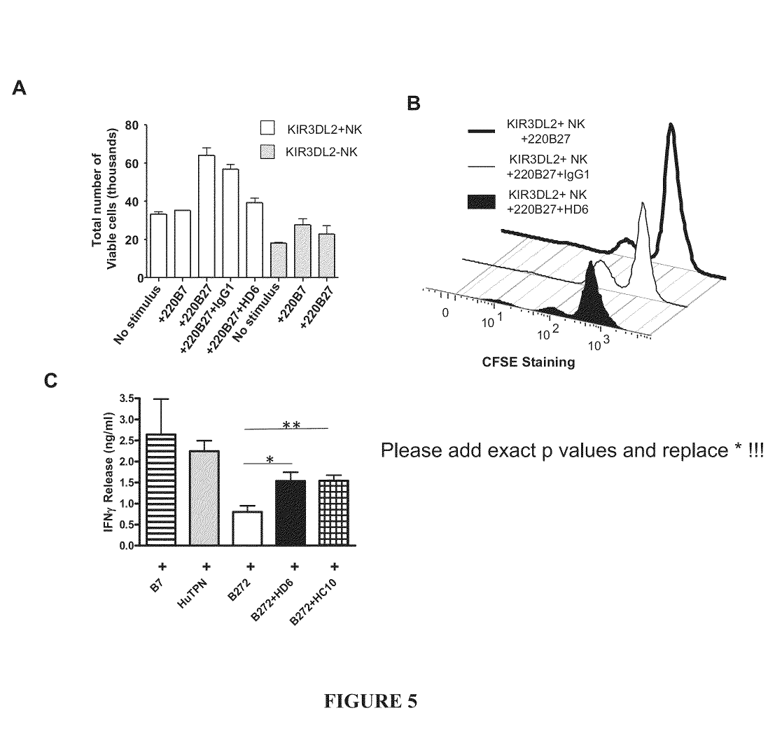

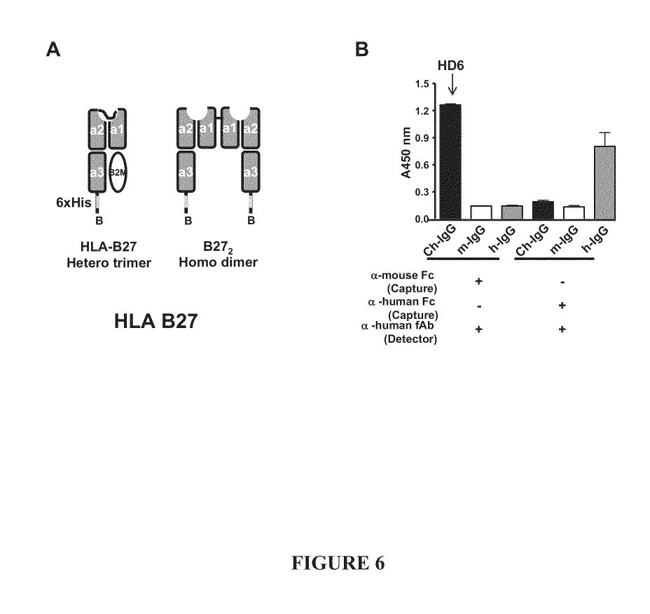

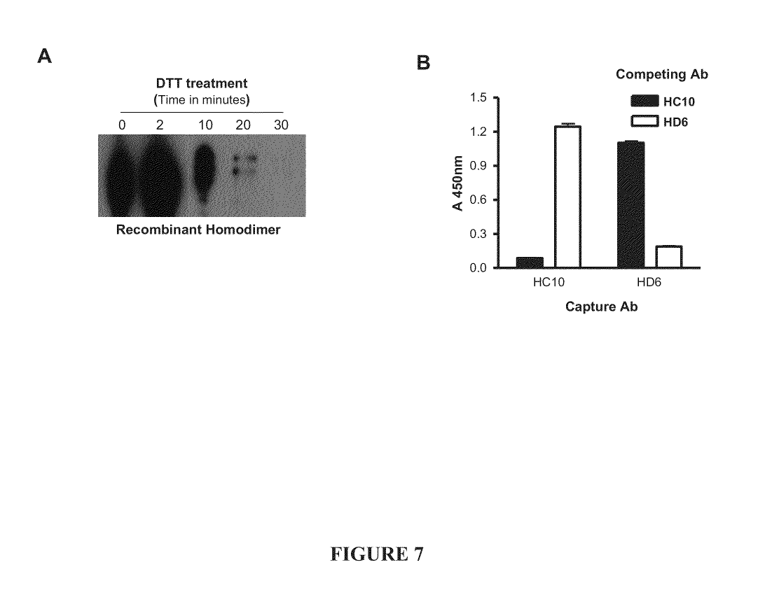

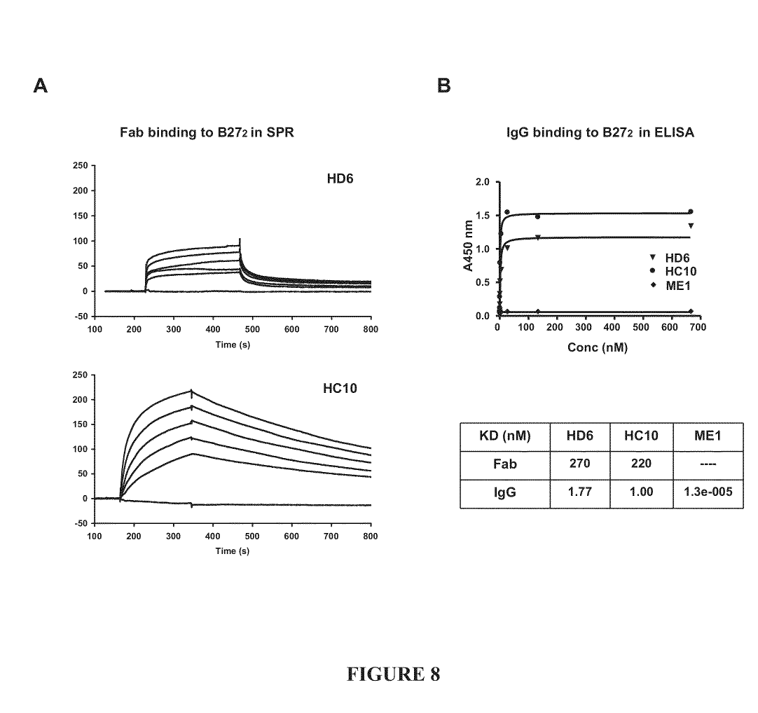

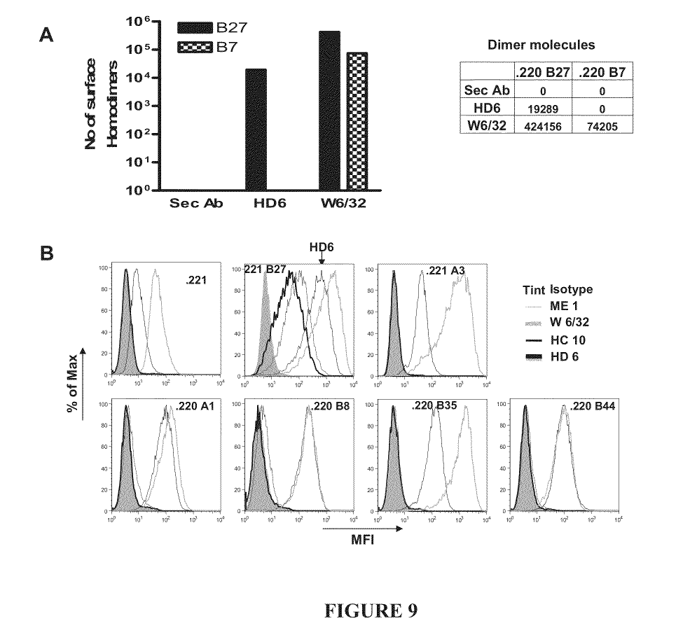

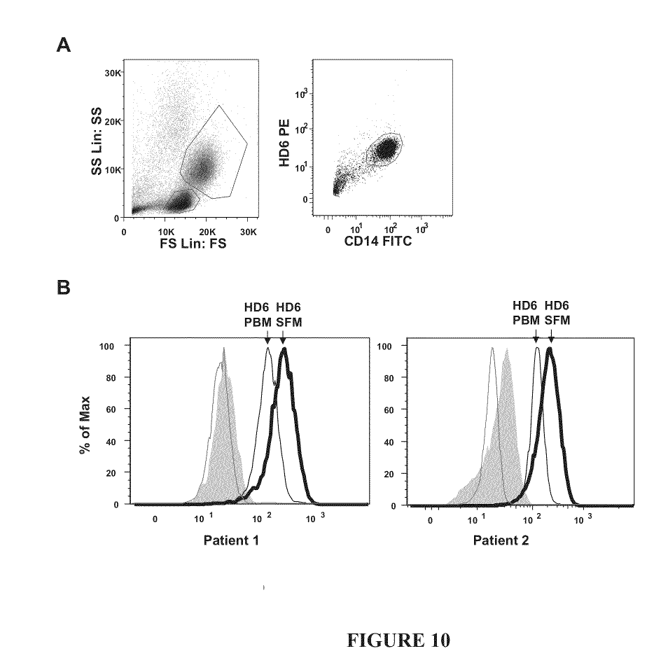

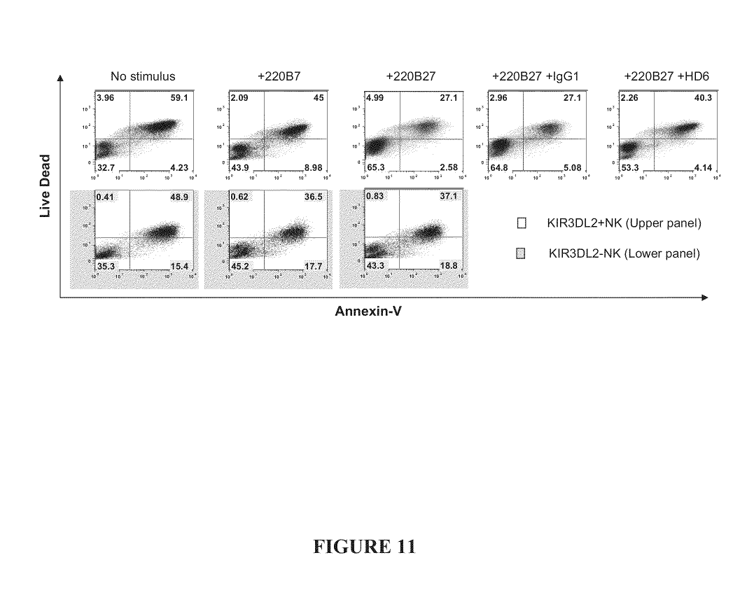

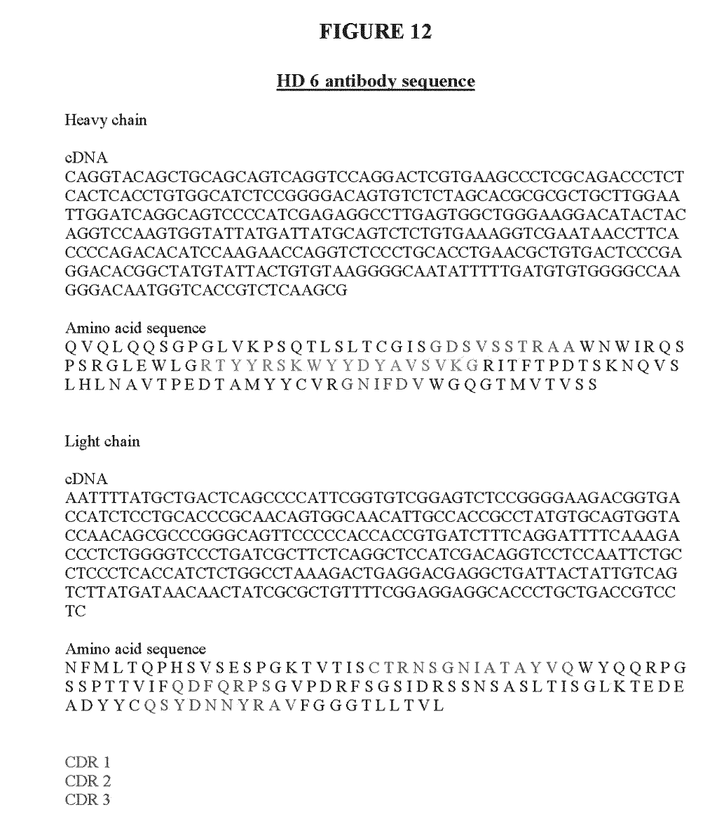

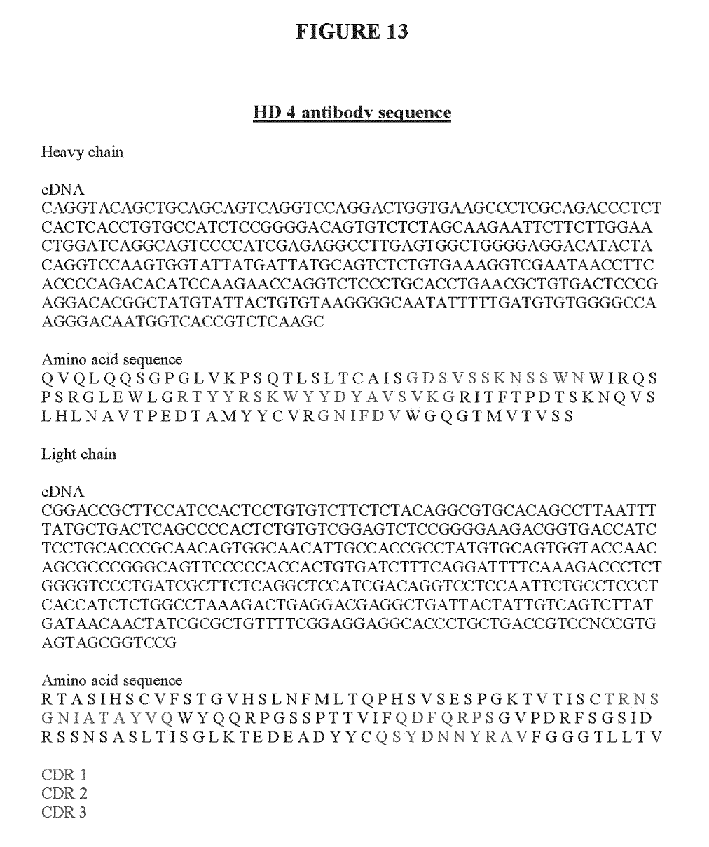

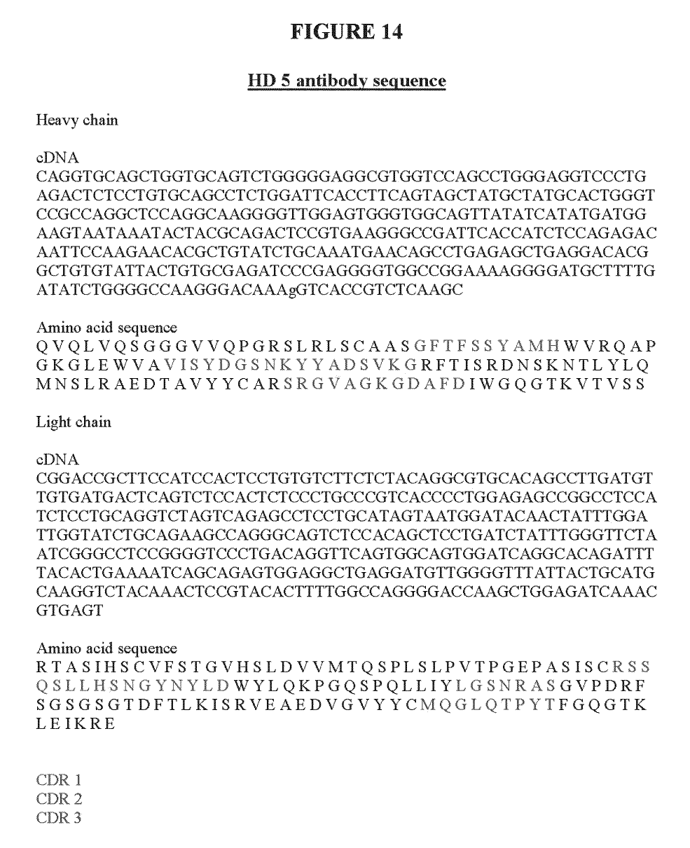

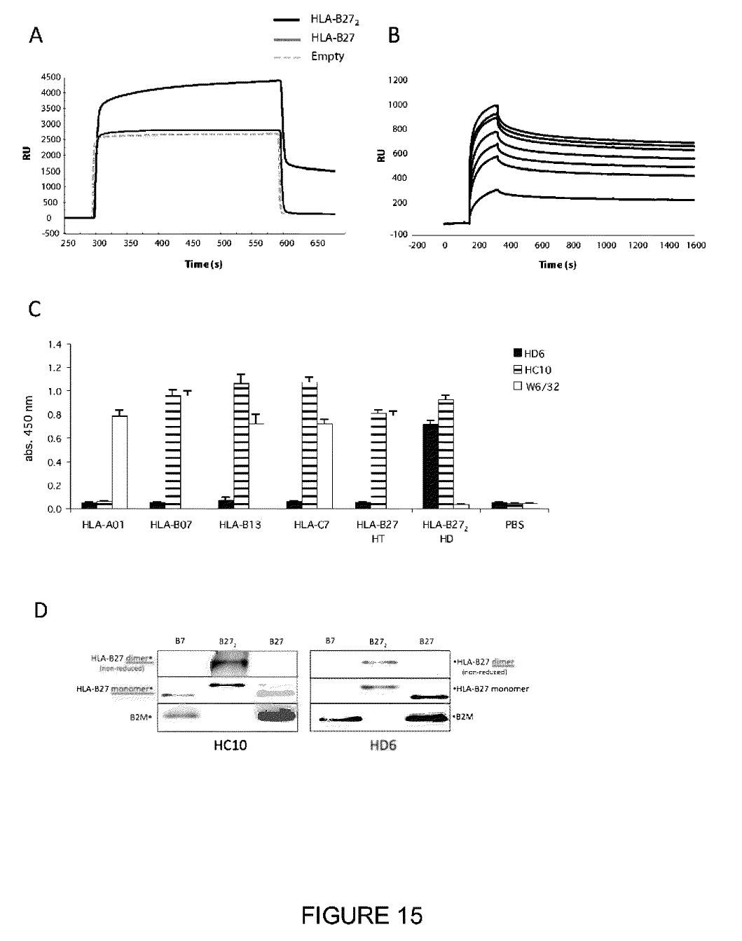

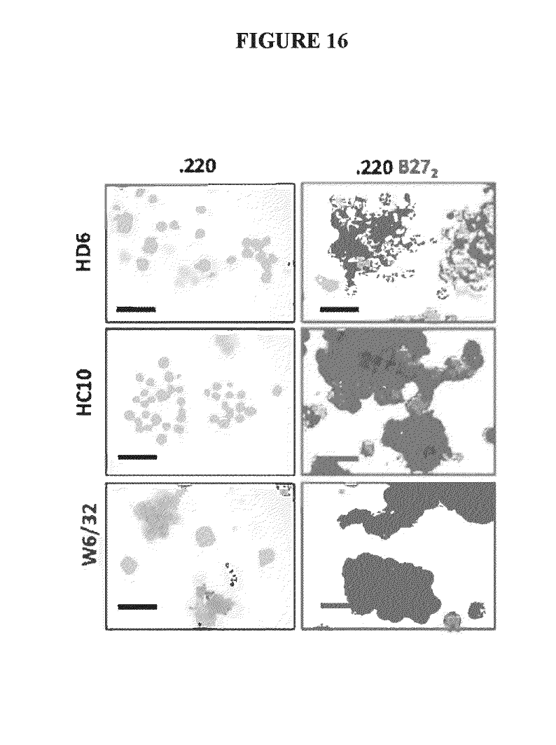

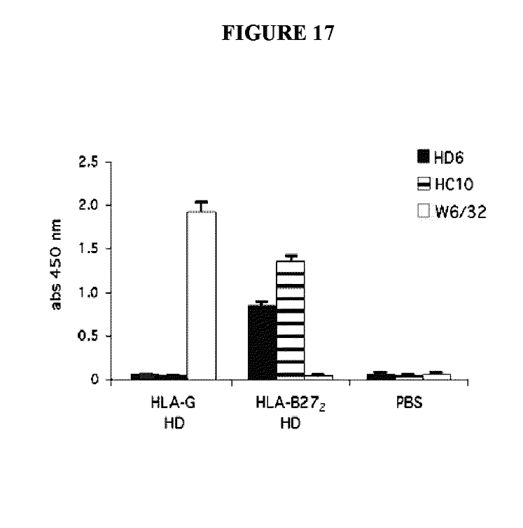

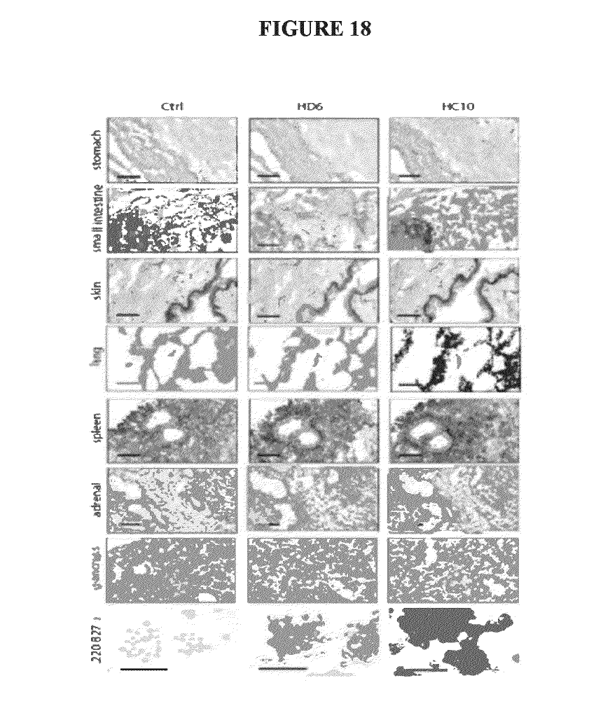

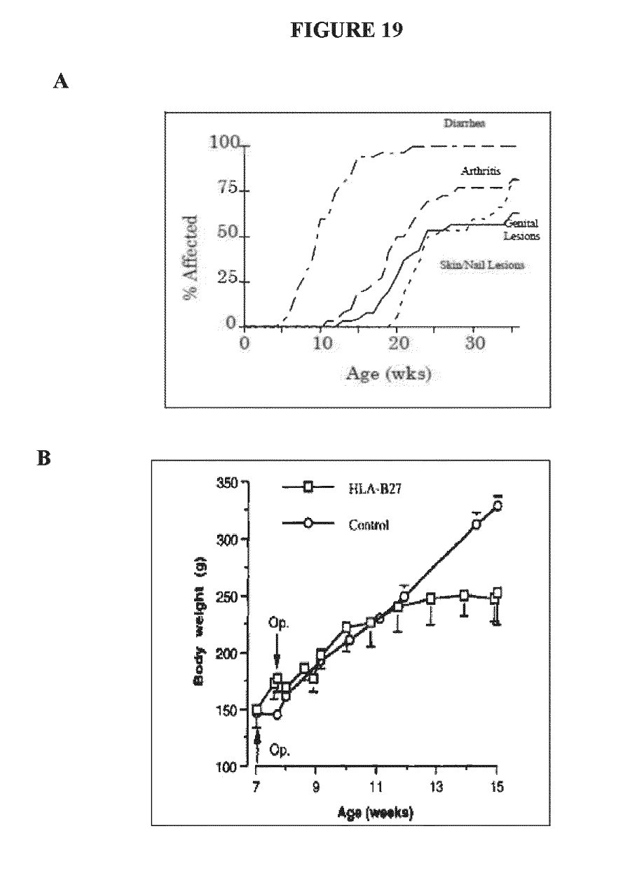

We next asked if HD6 recognized cell surface-expressed B27₂, which are subjected to post-translational modification. We first studied LBL721.220 B-lymphocytic cells (henceforth shortened to 0.220) stably transfected with HLA-B27.220 lack functional tapasin and express high levels of surface B27₂ as a consequence of endosomal recycling of unstable heterotrimers from the cell surface (10). As controls, 0.220 transfected with HLA-B7, B27-C67S (mutation of cysteine 67 to serine abrogates cell surface homodimer expression (10)) and B27 super-transfected with human tapasin (B27 HuTPN) were used. Super-transfection of HuTPN reduces B27₂ expression and increases levels of heterotrimer expression (10). HD6 bound strongly to 0.220 B27 cells, with reduced binding to HuTPN cells, consistent with their reduced levels of cell surface homodimer (FIG. 2A). No significant binding to 0.220 B7 or C67S cells was observed. By contrast, both ME1 and HC10 bound to all cell lines, indicating the presence of both HLA heterotrimers and free heavy chains, respectively. Semi-quantitative measurement revealed approx. 20,000 HD6-reactive surface molecules on 0.220B27 cells (FIG. 9A). HD6 also stained other B27-transfected cell lines, including 721.221 HLA-B27 (FIG. 9B), but not to 0.221A1 or 0.220A3, B8, B35 or B44 (FIG. 9B). Immunoprecipitation using the human monocytic cell line U937 transduced with a lentivirus expressing HA-tagged HLA-B27 confirmed B27₂ binding to HD6. A B27 band with approximate molecular weight 90 kDa (dimer form) in the absence of DTT that reduced to a single band of approx 50 kDa under reducing conditions was precipitated by HD6 (FIG. 2B). We also observed a second band at 45 kDa under non-reducing conditions. This might represent a (cys67 independent) non-covalently linked B27 dimer or reactivity with a monomeric structure.

B27₂ Expression on Monocytes Differs Significantly Between HLA-B27+ve Healthy Individuals and AS Patients

We next asked whether HD6 could detect B27₂ expression on peripheral blood mononuclear cells (PBMC) of HLA-B27 genotyped healthy donors and AS patients by flow cytometry (FIG. 10A). Indeed, B27+ healthy individuals expressed low but clearly detectable levels of B27₂ on monocytes (FIG. 3A) and B-lymphocytes (FIGS. 3C and D) when compared to matched samples from B27− healthy controls. Moreover, B27₂ expression on monocytes from AS patients was significantly higher when compared to monocytes from B27+ (p=0.01) or B27− (p=0.006) healthy individuals, respectively (FIG. 3B). In addition, B27₂ expression was also confirmed on synovial fluid (SF) monocytes from two patients with SpA. For these two patients, binding to SF monocytes appeared slightly greater than for matched peripheral blood monocytes (FIG. 10B). T and NK cells from AS patients (and controls) were consistently negative for B27₂ expression by HD6 staining.

HD6 Inhibits B27₂ Binding to KIR3DL1, KIR3DL2 and LILRB2 Receptors

Since we have previously proposed that B27₂ may contribute to SpA pathogenesis by binding Killer Inhibitory Receptors (KIR) and/or Leukocyte Immunoglobulin-like Receptor (LILR) receptors (14), we next asked if HD6 could inhibit these interactions. Indeed, HD6 reduced B27₂ tetramer binding to murine Baf3 cells stably transfected with either KIR3DL1, KIR3DL2 or LILRB2 (FIG. 4A, upper three panels). The inhibitory effect was most pronounced for homodimer tetramer binding to disease associated KIR3DL2 (which does not bind B27 heterotrimers), both for HD6 and HC10 antibody (FIG. 4B). IgG 1 isotype control antibody did not affect homodimer tetramer binding. As expected, HD6 did not interfere with HLA-B27 heterotrimer tetramer binding to KIR3DL1 or to LILRB2 (FIG. 4A, lower panels). To confirm specificity, we also studied HLA-A3, a natural ligand for KIR3DL2. HD6 did not interfere with HLA-A3 tetramer binding to KIR3DL2 and LILRB2 receptors.

HD6 Inhibits the Effects of Co-Culture of KIR3DL2+ Human NK Cells with B27₂ Expressing Cells (Protection from Apoptosis and Inhibition of IFNγ Production)

We have previously shown that co-culture of primary human KIR3DL2+ NK cells with 0.220 B27 cells (expressing B27₂) resulted in reduced apoptosis and enhanced survival (26). This anti-apoptotic effect was also observed if the human hYT cell line was transduced with KIR3DL2 (FIG. 5A upper panels). The effect was significantly reduced (P=0.0039) close to 0.220B7 control levels if HD6 was pre-incubated with 0.220 B27 cells and present in cell culture (FIG. 5A). Moreover, the total number of surviving KIR3DL2+ cells, which was increased in the presence of 0.220 B27 cells, was reduced by the presence of HD6 (FIG. 5B). Finally, IFN□ production in KIR3DL2+ human NK cells, reduced by co-culture with 0.220 B27 cells, could be partially restored by HD6 (or HC10) pre-incubation (FIG. 5C). Similar effects were observed for KIR3DL2-transduced Jurkat cells (data not shown).

Discussion

Using a novel, B27₂-specific antibody we have formally demonstrated significantly increased B27₂ expression on blood monocytes from patients with Ankylosing Spondylitis. Furthermore, in healthy individuals, possession of the HLA-B27 allele correlates with low-level expression of B27₂ on B-lymphocytes. HD6 inhibited binding of B27₂ to KIR3DL1, KIR3DL2 and LILRB2 immunoreceptors and blocked B27₂-mediated survival and proliferation of human NK cells expressing KIR3DL2. The HD6-specific functional inhibition of B27₂ binding to KIR3DL2 is of particular significance since AS patients' NK and T cells are enriched for expression of this receptor (26), which is not a ligand for “normal” HLA-B27 heterotrimers. Indeed KIR3DL2 is a marker for a population of IL17-producing cells in AS patients that can be expanded and driven to produce IL17 by B27₂-expressing cells (data not shown). Finally our data suggest that the epitope recognized by HD6 is directly involved in this receptor interaction. This antibody thus serves as a powerful tool to study the formation, interactions and potential pathogenic role of B27₂ homodimer in Ankylosing Spondylitis and other HLA-B27 associated Spondyloarthropathies. In addition, the B27₂ specific antibody provides a means to intervene or modulate B27-mediated diseases and pathologies and may be used to treat disease.

Until now, no B27₂ specific reagent existed and, therefore, investigation of B27₂ expression has been limited. Specific and precise detection and assessment, or even modulation, of B27₂ has not been possible. HD6 antibody demonstrates several clear differences in binding behaviour compared to HC10, the only existing antibody that consistently binds to B27 β2m free heavy chains (6;14). Firstly, HC10 is not B27₂ specific and recognizes additional HLA-B, C and A alleles (20) not seen by HD6. Secondly, HD6 and HC10 appeared to recognize different B27₂ epitopes, since DTT treatment abrogated HD6 recognition and, finally, the antibodies did not compete for binding. The HC10 binding site has been mapped to a linear but possibly discontinuous epitope in the region of the α1 chain residues P57-R62 (21). Our data would suggest that HD6 recognizes an epitope generated as a result of Cys 67 disulfide bonding of two B27 heavy chains.

Previous studies using HC10 have demonstrated that AS patients express more HLA class I heavy chains on their monocytes than controls (23). Dimers have also been detected in dendritic cells of HLA-B27+ individuals after activation with LPS (12). However it as not previously possible to demonstrate cell surface B27₂ expression either in HLA-B27+ healthy individuals or AS patients.

In HLA-B27+ AS patients, B27₂ expression on monocytes, was significantly increased compared to both B27+ and − healthy controls. Thus B27₂ expression is quantitatively and/or qualitatively different in AS patients when compared to healthy controls. Notably T or NK cells are either B27₂ negative or expression levels are below the detection limit of HD6. We postulate that the possession of the HLA-B27 allotype leads to low-level B27₂ expression restricted to blood monocytes (and B-lymphocytes, where low but statistically significant expression was detected in AS patients and B27+ controls). Thus B27₂ expression appears to be cell-type specific, and our data suggest that monocytes may play a key role in disease. Furthermore slightly higher B27₂ levels could be detected on synovial monocytes. The availability of B27₂ specific antibody now makes it possible to address factors regulating B27₂ expression, and to prospectively quantify B27₂ levels on different cell types and to correlate expression with AS disease activity. These studies are of great importance given that only a minority of HLA B27-positive individuals develop AS and that diagnosis is often delayed (28).

Co-culturing B27₂ expressing cells with KIR3DL2+ NK cells from AS patients blocks NK cell apoptosis and increases NK cell proliferation (26). This phenomenon mimics the in-vivo situation where KIR3DL2 expressing NK and CD4 T cells are expanded in the periphery and synovial fluid of patients with B27 associated arthritis (26, and data not shown). The lack of association of spondylarthritis with other KIR3DL2 binding alleles such as HLA-A3 and A11 suggests that expansion of KIR3DL2 expressing lymphocytes may result from unique properties of the interaction with B27₂. Indeed HLA-A3 does not have the same effects as on KIR3DL2+ T cells ex vivo (data not shown). It is therefore of great importance that HD6 blocked the interaction of B27₂ with KIRs and restored the normal cellular phenotype of KIR3DL2+ NK cells by increasing apoptosis and IFN gamma production and reducing proliferation. Thus, HD6 could potentially be used to specifically target the B27₂-KIR3DL2 interaction in AS patients while not impacting on other HLA class I molecules and their respective functions.

In summary, we describe a novel phage display-derived monoclonal antibody that recognizes an aberrant and potentially pathogenic HLA-B27 dimer expressed on the monocytes of patients with Ankylosing Spondylitis. HD6 specifically inhibits immunoreceptor recognition of B27₂, and will be a powerful investigative and potentially therapeutic tool in SpA treatment.

### REFERENCES

(1) Brewerton D A, Hart F D, Nicholls A, Caffrey M, James D C, Sturrock R D. Ankylosing spondylitis and HL-A 27. Lancet 1973; 1(7809):904-907.
(2) Brown M A, Pile K D, Kennedy L G, Calin A, Darke C, Bell J et al. HLA class I associations of ankylosing spondylitis in the white population in the United Kingdom. Ann Rheum Dis 1996; 55(4):268-270.
(3) Gaston H. Mechanisms of Disease: the immunopathogenesis of spondyloarthropathies. Nat Clin Pract Rheumatol 2006; 2(7):383-392.
(4) Allen R L, Bowness P, McMichael A. The role of HLA-B27 in spondylarthritis. Immunogenetics 1999; 50(3-4):220-227.
(5) Madden D R. The three-dimensional structure of peptide-MHC complexes. Annu Rev Immunol 1995; 13:587-622.
(6) Allen R L, O'Callaghan C A, McMichael A J, Bowness P. Cutting edge: HLA-B27 can form a novel beta 2-microglobulin-free heavy chain homodimer structure. J Immunol 1999; 162(9):5045-5048.
(7) Mear J P, Schreiber K L, Munz C, Zhu X, Stevanovic S, Rammensee H G et al. Misfolding of HLA-B27 as a result of its B pocket suggests a novel mechanism for its role in susceptibility to spondyloarthropathies. J Immunol 1999; 163(12):6665-6670.
(8) Dangoria N S, DeLay M L, Kingsbury D J, Mear J P, Uchanska-Ziegler B, Ziegler A et al. HLA-B27 misfolding is associated with aberrant intermolecular disulfide bond formation (dimerization) in the endoplasmic reticulum. J Biol Chem 2002; 277(26):23459-23468.
(9) Antoniou A N, Ford S, Taurog J D, Butcher G W, Powis S J. Formation of HLA-B27 homodimers and their relationship to assembly kinetics. J Biol Chem 2004; 279(10):8895-8902.
(10) Bird L A, Peh C A, Kollnberger S, Elliott T, McMichael A J, Bowness P. Lymphoblastoid cells express HLA-B27 homodimers both intracellularly and at the cell surface following endosomal recycling. Eur J Immunol 2003; 33(3):748-759.
(11) Capps G G, Robinson B E, Lewis K D, Zuniga M C. In vivo dimeric association of class I MHC heavy chains. Possible relationship to class I MHC heavy chain-beta 2-microglobulin dissociation. J Immunol 1993; 151(1):159-169.
(12) Powis S J, Santos S G, Antoniou A N. Biochemical features of HLA-B27 and antigen processing. Adv Exp Med Biol 2009; 649:210-216.
(13) Kollnberger S, Chan A, Sun M Y, Chen L Y, Wright C, di Gleria K et al. Interaction of HLA-B27 homodimers with KIR3DL1 and KIR3DL2, unlike HLA-B27 heterotrimers, is independent of the sequence of bound peptide. Eur J Immunol 2007; 37(5):1313-1322.
(14) Kollnberger S, Bird L, Sun M Y, Retiere C, Braud V M, McMichael A et al. Cell-surface expression and immune receptor recognition of HLA-B27 homodimers. Arthritis Rheum 2002; 46(11):2972-2982.
(15) Kollnberger S, Bowness P. The role of B27 heavy chain dimer immune receptor interactions in spondylarthritis. Adv Exp Med Biol 2009; 649:277-285.
(16) Kollnberger S, Bird L A, Roddis M, Hacquard-Bouder C, Kubagawa H, Bodmer H C et al. HLA-B27 heavy chain homodimers are expressed in HLA-B27 transgenic rodent models of spondylarthritis and are ligands for paired Ig-like receptors. J Immunol 2004; 173(3):1699-1710.
(17) Taurog J D. Animal models of spondylarthritis. Adv Exp Med Biol 2009; 649:245-254.
(18) Taurog J D, Dorris M L, Satumtira N, Tran T M, Sharma R, Dressel R et al. Spondylarthritis in HLA-B27/human beta2-microglobulin-transgenic rats is not prevented by lack of CD8. Arthritis Rheum 2009; 60(7):1977-1984.
(19) Khare S D, Hansen J, Luthra, H S, David, C S. HLA-B27 heavy chains contribute to spontaneous inflammatory disease in B27/human βm double transgenic mice with disrupted βm. J Clin Invest. 1996 15:2746-2755.
(20) Stam N J, Spits H, Ploegh H L. Monoclonal antibodies raised against denatured HLA-B locus heavy chains permit biochemical characterization of certain HLA-C locus products. J Immunol 1986; 137(7):2299-2306.
(21) Perosa F, Luccarelli G, Prete M, Favoino E, Ferrone S, Dammacco F. Beta 2-microglobulin-free HLA class I heavy chain epitope mimicry by monoclonal antibody HC-10-specific peptide. J Immunol 2003; 171(4):1918-1926.
(22) Raine T, Brown D, Bowness P, Hill Gaston J S, Moffett A, Trowsdale J et al. Consistent patterns of expression of HLA class I free heavy chains in healthy individuals and raised expression in spondyloarthropathy patients point to physiological and pathological roles. Rheumatology (Oxford) 2006; 45(11):1338-1344.
(23) Tsai W C, Chen C J, Yen J H, Ou T T, Tsai J J, Liu C S et al. Free HLA class I heavy chain-carrying monocytes—a potential role in the pathogenesis of spondyloarthropathies. J Rheumatol 2002; 29(5):966-972.
(24) Held G, Matsuo M, Epel M, Gnjatic S, Ritter G, Lee S Y et al. Dissecting cytotoxic T cell responses towards the NY-ESO-1 protein by peptide/MHC-specific antibody fragments. Eur J Immunol 2004; 34(10):2919-2929.
(25) Hoogenboom H R, Lutgerink J T, Pelsers M M, Rousch M J, Coote J, Van Neer N et al. Selection-dominant and nonaccessible epitopes on cell-surface receptors revealed by cell-panning with a large phage antibody library. Eur J Biochem 1999; 260(3):774-784.
(26) Chan A T, Kollnberger S D, Wedderburn L R, Bowness P. Expansion and enhanced survival of natural killer cells expressing the killer immunoglobulin-like receptor KIR3DL2 in spondylarthritis. Arthritis Rheum 2005; 52(11):3586-3595.
(27) Allen R L, Raine T, Haude A, Trowsdale J, Wilson M J. Leukocyte receptor complex-encoded immunomodulatory receptors show differing specificity for alternative HLA-B27 structures. J Immunol 2001; 167(10):5543-5547.
(28) Sieper J. Developments in the scientific and clinical understanding of the spondyloarthritides. Arthritis Res Ther 2009; 11(1):208.

### Example 2

Further studies were performed to characterize the HD6 antibody and its binding specificity.

HD6 Binds to Recombinant B27₂ and Differs in Specificity from HC10 and W6/32 Antibodies

The HD6 antibody showed high specificity to HLA-B27₂ homodimers when compared to HLA-B27 heterotrimers in Surface Plasmon Resonance (SPR) (FIG. 15A). A high avidity (KD=2.8 nM) of HD6 to HLA-B27₂ was measured using different concentrations of HD6 flowed over the immobilized HLA-B27₂ (FIG. 15B). We compared the specificity of HD6 with a panel of antibodies recognizing HLA-Class 1 molecules, including HC10, known to bind β2m-free heavy chains and HLA-B27₂ dimers (Stam, N J et al (1986) J Immunol 137, 2299-2306), and the W6/32 antibody known to bind a broad class of HLA-Class I molecules, but not β2m-free heavy chains nor HLA-B27₂ dimers. HD6 binds to recombinant HLA-B27₂ complexes but not to HLA-A1, B7, B13, C7 or B27 complexes in ELISA (FIG. 15C). HC10 also recognized HLA-B27₂ as described previously (Allen, R L et al (1999). J Immunol 162, 5045-5048) but, additionally, binds to other complexes including HLA-B7, B13 & C7. By contrast, the W6/32 antibody recognized only heterotrimeric forms of HLA-A I, B7, B 13, C7 and B27, but not the homodimeric form of B27₂ that lacks β2m (FIG. 15C).

HD6 Epitope and Binding Studies

The HD6 epitope seems to be present within the HLA-B27 heavy chain sequence and is at least partially linear since Western Blot analysis revealed a binding signal after DTT treatment of HLA-B27 heterotrimeric and HLA-B27₂ homodimer complexes, respectively (FIG. 15D). These data suggest that the epitope of HD6 is masked by β2m within a properly folded HLA-B27 heterotrimeric complex and is only accessible after a conformational modification of the HLA backbone leading to a disruption of β2m binding. HD6 antibody is HLA-B27 sequence specific and not just binding to any HLA class-I homodimer as demonstrated by experiments using recombinant HLA-G homodimers by ELISA (FIG. 17).

Immunohistochemistry with Cells and Human Tissue

Immunocytochemistry (ICC) of LBL721.220 B lymphocytic cells transfected with HLA-B27 (0.220 B27 cells) confirm the binding of HD6 and HD10 to cell surface expressed HLA-B27₂ homodimers (FIG. 16). The W6/32 control antibody binds to 0.220 B27₂ cells indicating the presence of HLA-B27 heterotrimers in the cell surface (FIG. 16). HD6 demonstrates restricted target specificity in human tissue by immunohistochemistry (IHC) since no specific binding to a broad panel of frozen normal tissues is detected (FIG. 18). This is in clear contrast to HD10 where cross-reactivity to different tissues especially of the lymphoid lineage (spleen) could be seen.

Materials and Methods

Methods and materials are as described below and/or in the prior example(s).

Patients and Samples

Ten ml heparinised venous blood was obtained from patients with AS/SpA and Rheumatoid Arthritis attending the Nuffield Orthopaedic Centre, and from healthy controls, with informed consent and appropriate ethical permission (COREC 06/Q1606/139).

Monoclonal Antibodies

KIR3DL1-(DX9, mouse IgG1) and KIR3DL2-specific (DX31, mouse IgG2a) mAbs were a gift from Jo Phillips (DNAX Palo Alto, Calif.). HC10 (IgG2a), which recognizes β2m-free class I heavy chains was a gift from Dr. Hidde Ploegh (MIT, MA). W6/32 (IgG2a, Dako, UK) recognizes human HLA class I heavy chains associated with β2m. ME1 recognizes HLA-B27, B7, B42, B67, B73, and Bw22. Antibodies were purified from hybridoma supernatants by protein-A sepharose. Rat anti-HA tag antibody (clone 3F10, Roche, UK) was used for Western Blotting. Isotype control mAb mouse IgG2a or IgG1 were purchased (Becton Dickinson, UK).

HLA Complexes

HLA-B*2705 homodimer and heterotrimer complexes were prepared as described previously (Kollnberger, S et al (2007) Eur J Immunol 37, 1313-1322). Briefly, recombinant HLA-B27 was expressed in E. Coli recA-BL21 (DE3) pLysS (GOLD) (Stratagene, UK), purified on Ni-NTA resin (Fast-Flow; Amersham Pharmacia Biotech, Little Chalfont, UK) and refolded by limiting dilution with or without β2m in the presence of Influenza Nucleoprotein NP383-391 peptide epitope SRYWAIRTR (SEQ ID NO: 36) or EBV EBNA3C epitope RRIYDLIEL (SEQ ID NO: 37) (Kollnberger, S et al (2007) Eur J Immunol 37, 1313-1322). Monomeric and dimeric forms were purified by FPLC purification and confirmed by non-reducing and reducing SDS-PAGE. After biotinylation, phycoerythrin (PE) labeled extravidin (Sigma, Poole, UK) was used to prepare tetramer complexes. Control HLA heterotrimeric complexes were refolded with the following peptide epitopes: HLA-A*0201 (“HLA-A2”) with SLYNTVATL (SEQ ID NO: 31), HLA-A*0301 with RLRAEAQVK (SEQ ID NO: 32), HLA-A*2401 with RYPLTFGW (SEQ ID NO: 33), or HLA-B*0702 (“HLA-B7”) with LPFDKTUM (SEQ ID NO: 34) or RPMTYKAAL (SEQ ID NO: 35) as described (Kollnberger, S et al (2002 Arthritis Rheum 46, 2972-2982 (November, 2002).

Surface Plasmon Resonance

Surface Plasmon Resonance (SPR) measurements were performed using a Biacore 3000. 240 response units (RU) of biotinylated HLA-B27₂ was immobilized on a streptavidin-coated chip (in flow cell 2). HD6 was injected over flow cells 1 and 2 at increasing concentrations from 0 μg/ml to 200 μg/ml. The flow cell was regenerated by injecting glycine pH 2.5 after each HD6 injection. Experiments were performed three times at 25° C. and Kd values were obtained after subtraction of background from flow cell 1.

ELISA Assays

Indirect Enzyme Linked Immuno Sorbent Assays (ELISA) for specificity was performed using plate-bound HLA complexes. Maxisorp (Nunc, Switzerland) 96 well plates were coated with 2 μg/ml biotinylated BSA, streptavidin (10 μg/ml) (Promega, Switzerland). Biotinylated HLA complexes were incubated for 1 h and wells were blocked with 5% Milk powder-PBS solution. Antibodies were allowed 1 h for binding. Following three washes with 0.3% PBS-T, anti-M13 (GE/Amersham, Switzerland) or anti-human Fab (Sigma, Switzerland) or anti-mouse Fc (BioRad, Switzerland), HRP-conjugated antibodies were used as detectors. Tetra-methylbenzidine was used as substrate for color development. 2N H₂SO₄ was used to stop the reaction and absorbance was read at 450 nm (Wallac Victor 2, Perkin-Elmer, Switzerland). Competition ELISA was performed using 5 μg/ml of either HD6 or HC10 antibodies coated on maxisorp 96 well plates. Following blocking of the wells with 5% milk powder in PBS, 1 μg/ml of B27₂ and 10 μg/ml competing antibody was incubated in triplicates for 1 h at RT. Streptavidin HRP (Roche, Switzerland) was incubated at 1:2,000 dilution for an additional hour. Upon stringent washes with 0.1% Tween20-PBS, wells were developed as described above in ELISA method.

Immunohistochemistry

For detection of the HLA-B27₂ in LBL721.220 cells and in frozen tissue sections, we used as primary antibodies HD6, HC10, W6/32 and Isotype control mAb mouse, and as secondary antibody a peroxidase anti-mouse Fc, followed by peroxidase treatment M.O.M (Vector Laboratories), and revealed with the 3,3′-diaminobenzidine (DAB) (Pierce).

Protein Analysis

Purified complex from HLA-B27, HLA-B27₂ & HLA-B8, HLA-B27-C67S were tested by Western blot using sodium dodecyl sulfate polyacrylamide electrophoresis and transferred onto nitrocellulose membranes, where they were probed with HD6 & HC10 antibodies. A secondary antibody peroxidase conjugated against mouse-Fc (Jackson) was used to reveal the presence of positive bands.

This invention may be embodied in other forms or carried out in other ways without departing from the spirit or essential characteristics thereof. The present disclosure is therefore to be considered as in all aspects illustrated and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

1. An isolated antibody or fragment thereof which recognizes HLA-B27 heavy-chain homodimers, B27₂, and which does not recognize or bind HLA-B27 heterotrimers (B27) including HLA-B27 heterotrimers with β2 microglobulin and peptide, wherein the antibody or fragment has:
(a) a heavy chain variable domain comprising the CDR1, CDR2 and CDR3 region sequences as set forth in FIG. 12 (SEQ ID NOS: 3-5), and a light chain variable domain comprising the CDR1, CDR2 and CDR3 region sequences as set forth in FIG. 12 (SEQ ID NOS: 8-10);
(b) a heavy chain variable domain comprising the CDR1, CDR2 and CDR3 region sequences as set forth in FIG. 13 (SEQ ID NOS: 13-15), and a light chain variable domain comprising the CDR1, CDR2 and CDR3 region sequences as set forth in FIG. 13 (SEQ ID NOS: 18-20); or
(c) a heavy chain variable domain comprising the CDR1, CDR2 and CDR3 region sequences as set forth in FIG. 14 (SEQ ID NOS: 23-25), and a light chain variable domain comprising the CDR1, CDR2 and CDR3 region sequences as set forth in FIG. 14 (SEQ ID NOS: 28-30).

2. The antibody of claim 1 which is a monoclonal antibody.

3. The isolated antibody or fragment of claim 1 which is selected from HD4, HD5 and HD6 antibody or an active fragment thereof, and has:
(a) a heavy chain variable domain having the amino acid sequence of HD-6 as set forth in FIG. 12 (SEQ ID NO: 2), and a light chain variable domain having the amino acid sequence of HD-6 as set forth in FIG. 12 (SEQ ID NO: 7);
(b) a heavy chain variable domain having the amino acid sequence of HD-4 as set forth in FIG. 13 (SEQ ID NO: 12), and a light chain variable domain having the amino acid sequence of HD-4 as set forth in FIG. 13 (SEQ ID NO: 17); or
(c) a heavy chain variable domain having the amino acid sequence of HD-5 as set forth in FIG. 14 (SEQ ID NO: 22), and a light chain variable domain having the amino acid sequence of HD-5 as set forth in FIG. 14 (SEQ ID NO: 27).

4. The isolated antibody or fragment of claim 1 which is an antibody or antibody fragment comprising a heavy chain and a light chain variable region comprising an amino acid sequence selected from the amino acid sequence set out in FIG. 12, FIG. 13 or FIG. 14 or highly homologous variants thereof comprising 1 to 3 amino acid substitutions in one or more CDR region of FIG. 12, 13 or 14, wherein said variants retain B27₂ specific binding.

5. The isolated antibody or fragment of claim 1 which is an antibody or fragment thereof wherein said isolated antibody is the form of an antibody F(ab′)2, scFv fragment, diabody, triabody or tetrabody.

6. The isolated antibody or fragment of claim 1 further comprising a detectable or functional label.

7. The isolated antibody of claim 6, wherein said detectable or functional label is a covalently attached drug.

8. The isolated antibody of claim 6, wherein said label is a radiolabel.

9. An isolated nucleic acid which comprises a sequence encoding an antibody or fragment of claim 1.

10. The isolated nucleic acid of claim 9 which comprises a nucleic acid sequence encoding a heavy chain variable region, wherein the nucleic acid comprises SEQ ID NO: 1, 11 or 21, and a nucleic acid sequence encoding a light chain variable region, wherein the nucleic acid comprises SEQ ID NO: 6, 16 or 26.

11. A method of preparing an antibody or fragment as defined in claim 1 which comprises expressing the nucleic acid of claim 9 or 10 under conditions to bring about expression of said antibody or fragment, and recovering the antibody or fragment.

12. A method for diagnosing or monitoring an HLA-B27 mediated disease or condition in a mammal wherein said disease or condition is diagnosed or monitored by determining the presence and/or amount of HLA-B27 homodimer comprising:
A. contacting a biological sample from a mammal in which the presence of and HLA-B27 mediated disease or condition is suspected with the antibody or fragment of claim 1 under conditions that allow binding of HLA-B27 homodimer to said antibody to occur; and
B. detecting whether binding has occurred between HLA-B27 homodimer from said sample and the antibody or determining the amount of binding that has occurred said HLA-B27 homodimer from said sample and the antibody;
wherein the detection of binding indicates the presence of HLA-B27 homodimer in said sample and of an HLA-B27 mediated disease or condition in said mammal.

13. The method of claim 12 wherein the antibody comprises a heavy chain and light chain variable region comprising an amino acid sequence selected from the amino acid sequence set out in FIG. 12, 13 or 14, or highly homologous variants thereof comprising 1 to 3 amino acid substitutions in one or more CDR region of FIG. 12, 13 or 14, wherein said variants retain B27₂ specific binding.

14. The method of claim 12 for diagnosing or monitoring one or more disease or condition selected from ankylosing spondylitis (AS), reactive arthritis (ReA or Reiter's syndrome), sacroileitis associated with psoriasis, sacroileitis associated with inflammatory bowel disease, undifferentiated oligoarthropathy, anterior uveitis, aortic regurgitation together with cardiac conduction abnormality and enthesis-related juvenile idiopathic arthritis.

15. A kit for the diagnosis or prognosis of an HLA-B27 mediated disease in which HLA-B27 homodimer B27₂ is present, said kit comprising an antibody or fragment of claim 1, optionally with reagents and/or instructions for use.

16. (canceled)

17. (canceled)

18. A pharmaceutical composition comprising an antibody or fragment as defined in claim 1 and a pharmaceutically acceptable vehicle, carrier or diluent.

19. A kit for the treatment or modulation of an HLA-B27 mediated disease or condition characterized by the presence of HLA-B27 homodimer, comprising a pharmaceutical dosage form of the pharmaceutical composition of claim 18, and a separate pharmaceutical dosage form comprising an additional agent selected from the group consisting of immunomodulatory agents, anti-inflammatory agents and combinations thereof.

20. A method of treatment of an HLA-B27 mediated disease or condition selected from ankylosing spondylitis (AS), reactive arthritis (ReA or Reiter's syndrome), sacroileitis associated with psoriasis, sacroileitis associated with inflammatory bowel disease, undifferentiated oligoarthropathy, anterior uveitis, aortic regurgitation together with cardiac conduction abnormality and enthesis-related juvenile idiopathic arthritis in a mammal which comprises administering to said mammal an effective amount of an antibody or fragment as defined in claim 1.
